(19) United States
(12) Patent Application Publication
(10) Pub. No.: US 2022/0266045 A1
(43) Pub. Date: Aug. 25, 2022

(54) Therapy Electrode Mesh Interface for Wearable Cardiac Therapeutic Devices

(71) Applicant: ZOLL Medical Corporation, Chelmsford, MA (US)

(72) Inventors: Christopher L. Swenglish, Connellsville, PA (US); Wolfgang Philipps, Columbus, OH (US)

(21) Appl. No.: 17/676,396

(22) Filed: Feb. 21, 2022

Related U.S. Application Data
(60) Provisional application No. 63/152,626, filed on Feb. 23, 2021.

(51) Int. Cl. A61N 1/39; A61N 1/04

(52) U.S. Cl. CPC A61N 1/3968; A61N 1/046; A61N 1/0484; A61N 1/3904

(57) ABSTRACT

A wearable cardiac therapeutic device for improved skin comfort when worn by a patient includes a therapy electrode configured to deliver therapeutic electrical pulses to a patient's heart; and a support garment configured to support and hold the therapy electrode against the patient's body. The support garment includes a support pocket disposed on an inside surface of the support garment for supporting the therapy electrode; and a mesh interface formed as part of the support pocket. The mesh interface is configured to facilitate electrical contact between the therapy electrode and the patient's skin. The mesh interface includes a first surface oriented toward the therapy electrode; a second surface oriented toward the patient's skin; a plurality of dielectric fibers comprising a nonmetallic material; and a plurality of conductive fibers or particles. The plurality of dielectric fibers and the plurality of conductive fibers or particles are interspersed to form a plurality of conductive pathways extending through the mesh interface, the plurality of conductive pathways being configured to conduct the therapeutic electrical pulses through the mesh interface from the therapy electrode to the patient.

### CROSS REFERENCE TO RELATED APPLICATIONS

This application claims the benefit of U.S. Provisional Patent Application No. 63/152,626, filed Feb. 23, 2021, which is incorporated by reference herein in its entirety.

### FIELD OF THE DISCLOSURE

The present disclosure relates to a support garment for a wearable cardiac monitoring and therapeutic medical device, such as a wearable cardioverter defibrillator (WCD).

### BACKGROUND OF THE DISCLOSURE

When a patient is deemed at high risk of death from arrhythmias, such as ventricular fibrillation or ventricular tachycardia, electrical devices can be implanted so as to be readily available when treatment is needed. However, patients who have recently had a heart attack or are awaiting such an implantable device can be kept in a hospital where corrective electrical therapy is generally close at hand. Long-term hospitalization is frequently impractical due to its high cost, or due to the need for patients to engage in normal daily activities.

Wearable cardioverter defibrillators can help bridge the gap for patients who have recently experienced cardiac arrest, who are susceptible to heart arrhythmias and are at temporary risk of sudden death, and/or who are awaiting an implantable device. Support garments have been developed for supporting the components of such wearable cardioverter defibrillators, including the sensing and therapeutic energy delivery electrodes, such that the electrodes are properly positioned against the patient's skin. Such support garments may incorporate a material that acts as an interface between the therapeutic energy delivery electrodes and the patient's skin in order to conduct electrical pulses from the therapeutic energy delivery electrodes to the patient and to permit conductive gel released by the therapeutic energy delivery electrodes to contact the patient's skin.

### SUMMARY OF SOME OF THE EMBODIMENTS

Non-limiting examples of embodiments will now be described.

In an example, a wearable cardiac therapeutic device for improved skin comfort when worn by a patient is provided. The device can comprise: at least one therapy electrode configured to deliver transcutaneous therapeutic pulses to a patient's heart; and a support garment configured to support and hold the at least one therapy electrode against the patient's body. The support garment may comprise: at least one support pocket disposed on an inside surface of the support garment for supporting the at least one therapy electrode on the support garment; and a mesh interface formed as part of the at least one support pocket, the mesh interface configured to facilitate electrical contact between the at least one therapy electrode and the patient's skin. The mesh interface may comprise: a first surface oriented toward the at least one therapy electrode; a second surface oriented toward the patient's skin; a plurality of dielectric fibers comprising at least one nonmetallic material; and a plurality of conductive fibers or particles. The plurality of dielectric fibers and the plurality of conductive fibers or particles are interspersed to form a plurality of conductive pathways extending through the mesh interface from the first surface to the second surface, the plurality of conductive pathways being configured to conduct the transcutaneous therapeutic pulses through the mesh interface from the at least one therapy electrode to the patient.

The mesh interface may be configured to facilitate a transfer of conductive gel from the at least one therapy electrode to the patient's skin.

The mesh interface may be configured to provide a comfortable feel on the patient's skin and to wick moisture away from the patient's skin.

The mesh interface may be configured to transmit a falloff signal from the at least one therapy electrode to the patient's skin configured to determine that the at least one therapy electrode is correctly positioned on the patient's body.

The mesh interface may be configured to provide an electrical impedance of the plurality of conductive pathways extending through the mesh interface from the first surface to the second surface of between approximately 0.01Ω-5Ω, more particularly approximately 0.1Ω-2Ω, and more particularly approximately 0.25Ω-1.5Ω.

The plurality of conductive fibers or particles can comprise an impedance measure of between approximately 10-250 Ω/meter, more particularly approximately 20-150 Ω/meter, and more particularly approximately 30-130 Ω/meter.

The mesh interface can further comprise a plurality of openings extending through the mesh interface from the first surface to the second surface, the mesh interface being configured to facilitate transfer of conductive gel from the at least one therapy electrode to the patient's skin via the plurality of openings. The mesh interface may be configured to receive conductive gel from a plurality of holes in the at least one therapy electrode in an amount of between approximately 0.1 cubic-centimeter (cc) to 30 cc of conductive gel, more particularly approximately 0.5 cc to 20 cc, and more particularly approximately 0.9 cc to 10 cc. The conductive gel may be configured to provide a predetermined electrical impendence of the plurality of conductive pathways extending through the mesh interface from the first surface to the second surface of between approximately 0.01Ω-5Ω, more particularly approximately 0.1Ω-2Ω, and more particularly approximately 0.25Ω-1.5Ω.

The mesh interface may be configured to be porous to the conductive gel from a plurality of holes in the at least one therapy electrode to provide a predetermined electrical impendence of the plurality of conductive pathways extending through the mesh interface from the first surface to the second surface of between approximately 0.01Ω-5Ω, more particularly approximately 0.1Ω-2Ω, and more particularly approximately 0.25Ω-1.5Ω. The mesh interface can comprise a plurality of openings, the plurality of opening comprising approximately 2-1000 openings per square inch of the mesh interface, more particularly approximately 5-500 openings per square inch of the mesh interface, and more particularly approximately 10-100 openings per square inch of the mesh interface. The mesh interface can comprise a plurality of openings having an average diameter in a range of between approximately 0.005″-0.3″ (0.13 mm-7.6 mm), more particularly approximately 0.01″-0.2″ (0.25 mm-5.1 mm), and more particularly approximately 0.05″-0.1′ (1.3 mm-2.5 mm).

A thickness of the mesh interface from the first surface to the second surface may be approximately 0.005″-0.5″ (0.13 mm-12.7 mm), more particularly approximately 0.01″-0.25″ (0.25 mm-6.4 mm), and more particularly approximately 0.03″-0.1″ (0.76 mm-2.5 mm).

The dielectric fibers can comprise, at least in part, fusible fibers that are configured to shrink in volume relative to the conductive fibers or particles when the mesh interface is exposed to heat. The fusible fibers are configured to shrink in volume relative to the conductive fibers or particles when the mesh interface is exposed to heat resulting in the conductive fibers or particles expressing more relative to the dielectric fibers, whereby the plurality of conductive pathways extending through the mesh interface project from the first surface and the second surface of the mesh interface.

The plurality of dielectric fibers of the mesh interface can comprise a dielectric yarn and the plurality of conductive fibers or particles of the mesh interface can comprise a conductive yarn. The dielectric yarn and the conductive yarn are intertwined together to form the mesh interface.

The dielectric yarn can comprise a texturized nylon or cotton yarn and a fusible yarn.

The conductive yarn can comprise silver plated nylon yarn.

The mesh interface can comprise approximately 10%-60% by weight of conductive yarn, more particularly approximately 15%-40% by weight of conductive yarn, and more particularly approximately 20%-35% by weight of conductive yarn.

The mesh interface can comprise a plurality of intertwined structures of the dielectric yarn and the conductive yarn. According to an example, the plurality of intertwined structures comprises a pattern of at least three intertwined structures. The at least three intertwined structures can comprise: a plurality of courses of the dielectric yarn intertwined with each other in a tubular pattern structure; at least one course of the conductive yarn intertwined with the plurality of courses of the dielectric yarn in a 1×1 rib pattern structure; and at least one pointelle pattern structure of intertwined dielectric yarn. The dielectric yarn comprises a texturized nylon or cotton yarn and a fusible yarn arranged together in a plated yarn structure, and wherein the texturized nylon or cotton yarn forms an exterior of the plurality of courses of the tubular pattern structure and the fusible yarn forms an interior of the plurality of courses of the tubular pattern structure.

The at least one course of the conductive yarn in the 1×1 rib pattern structure may extend from the first surface of the mesh interface to the second surface of the mesh interface. The at least one 1×1 rib pattern structure may be configured such that the conductive yarn stands out of the first and second surfaces of the mesh interface. The fusible yarn may be configured to melt, dissipate, and/or shrink in volume relative to the conductive yarn when exposed to heat to cause the dielectric yarn to contract relative to the conductive yarn and enhance the standing out of the conductive yarn from the first and second surfaces of the mesh interface.

The at least one pointelle pattern structure may define a plurality of openings extending through the mesh interface from the first surface to the second surface, the mesh interface being configured to facilitate transfer of conductive gel from the at least one therapy electrode to the patient's skin via the plurality of openings.

The at least one nonmetallic material can comprise nylon or cotton.

The mesh interface may provide improved skin comfort as determined by a Human Skin Irritation Test (ISO 10993-10 C3.3).

In an example, a wearable cardiac therapeutic device for improved skin comfort when worn by a patient is provided. The device can comprises: at least one therapy electrode configured to deliver transcutaneous therapeutic pulses to a patient's heart; and a support garment configured to support and hold the at least one therapy electrode against the patient's body. The support garment can comprise: at least one support pocket disposed on an inside surface of the support garment for supporting the at least one therapy electrode on the support garment; and a mesh interface formed as part of the at least one support pocket. The mesh interface may be configured to be facilitate electrical contact between the at least one therapy electrode and the patient's skin. The mesh interface can comprise: a first surface oriented toward the at least one therapy electrode; a second surface oriented toward the patient's skin; a plurality of dielectric fibers comprising at least one nonmetallic material; a plurality of conductive fibers or particles; and a plurality of openings extending through the mesh interface from the first surface to the second surface. The mesh interface is configured to facilitate a transfer of conductive gel from the at least one therapy electrode to the patient's skin via the plurality of openings.

The mesh interface may be configured to provide a comfortable feel on the patient's skin and to wick moisture away from the patient's skin.

The mesh interface may be configured to transmit a falloff signal from the at least one therapy electrode to the patient's skin configured to determine that the at least one therapy electrode is correctly positioned on the patient's body.

The plurality of dielectric fibers and the plurality of conductive fibers or particles may be interspersed to form a plurality of conductive pathways extending through the mesh interface from the first surface to the second surface, the plurality of conductive pathways being configured to conduct the transcutaneous therapeutic pulses through the mesh interface from the at least one therapy electrode to the patient.

The mesh interface may be configured to provide an electrical impedance of the plurality of conductive pathways extending through the mesh interface from the first surface to the second surface of between approximately 0.01Ω-5Ω, more particularly approximately 0.1Ω-2Ω, and more particularly approximately 0.25Ω-1.5Ω.

The plurality of conductive fibers or particles can comprise an impedance measure of between approximately 10-250 Ω/meter, more particularly approximately 20-150 Ω/meter, and more particularly approximately 30-130 Ω/meter.

The mesh interface may be configured to receive conductive gel from a plurality of holes in the at least one therapy electrode in an amount of between approximately 0.1 cubic-centimeter (cc) to 30 cc of conductive gel, more particularly approximately 0.5 cc to 20 cc, and more particularly approximately 0.9 cc to 10 cc. The conductive gel may be configured to provide a predetermined electrical impendence of the plurality of conductive pathways extending through the mesh interface from the first surface to the second surface of between approximately 0.01Ω-5Ω, more particularly approximately 0.1Ω-2Ω, and more particularly approximately 0.25Ω-1.5Ω.

The mesh interface may be configured to be porous to the conductive gel from a plurality of holes in the at least one therapy electrode to provide a predetermined electrical impendence of the plurality of conductive pathways extending through the mesh interface from the first surface to the second surface of between approximately 0.01Ω-5Ω, more particularly approximately 0.1Ω-2Ω, and more particularly approximately 0.25Ω-1.5Ω. The plurality of openings can comprise approximately 2-1000 openings per square inch of the mesh interface, more particularly approximately 5-500 openings per square inch of the mesh interface, and more particularly approximately 10-100 openings per square inch of the mesh interface. The plurality of openings can have an average diameter in a range of between approximately 0.005″-0.3″ (0.13 mm-7.6 mm), more particularly approximately 0.01″-0.2″ (0.25 mm-5.1 mm), and more particularly approximately 0.05″-0.1″ (1.3 mm-2.5 mm).

A thickness of the mesh interface from the first surface to the second surface may be approximately 0.005″-0.5″ (0.13 mm-12.7 mm), more particularly approximately 0.01″-0.25″ (0.25 mm-6.4 mm), and more particularly approximately 0.03″-0.1″ (0.76 mm-2.5 mm).

The dielectric fibers can comprise, at least in part, fusible fibers that are configured to shrink in volume relative to the conductive fibers or particles when the mesh interface is exposed to heat. The fusible fibers may be configured to shrink in volume relative to the conductive fibers or particles when the mesh interface is exposed to heat resulting the conductive fibers or particles expressing more relative to the dielectric fibers, whereby the plurality of conductive pathways extending through the mesh interface project from the first surface and the second surface of the mesh interface.

According to an example, the plurality of dielectric fibers of the mesh interface can comprise a dielectric yarn and the plurality of conductive fibers or particles of the mesh interface comprise a conductive yarn. The dielectric yarn and the conductive yarn can be intertwined together to form the mesh interface. The dielectric yarn can comprise a texturized nylon or cotton yarn and a fusible yarn. The conductive yarn can comprise silver plated nylon yarn.

The mesh interface can comprise approximately 10%-60% by weight of conductive yarn, more particularly approximately 15%-40% by weight of conductive yarn, and more particularly approximately 20%-35% by weight of conductive yarn.

According to an example, the mesh interface can comprise a plurality of intertwined structures of the dielectric yarn and the conductive yarn. The plurality of intertwined structures can comprise a pattern of at least three intertwined structures. The at least three intertwined structures can comprise: a plurality of courses of the dielectric yarn intertwined with each other in a tubular pattern structure; at least one course of the conductive yarn intertwined with the plurality of courses of the dielectric yarn in a 1×1 rib pattern structure; and at least one pointelle pattern structure of intertwined dielectric yarn.

The dielectric yarn can comprise a texturized nylon or cotton yarn and a fusible yarn arranged together in a plated yarn structure. The texturized nylon or cotton yarn forms an exterior of the plurality of courses of the tubular pattern structure and the fusible yarn forms an interior of the plurality of courses of the tubular pattern structure.

The at least one course of the conductive yarn in the 1×1 rib pattern structure extends from the first surface of the mesh interface to the second surface of the mesh interface. The at least one 1×1 rib pattern structure may be configured such that the conductive yarn stands out of the first and second surfaces of the mesh interface. The fusible yarn may be configured to melt, dissipate, and/or shrink in volume relative to the conductive yarn when exposed to heat to cause the dielectric yarn to contract relative to the conductive yarn and enhance the standing out of the conductive yarn from the first and second surfaces of the mesh interface.

The at least one pointelle pattern structure may define a plurality of openings extending through the mesh interface from the first surface to the second surface, the mesh interface being configured to facilitate transfer of conductive gel from the at least one therapy electrode to the patient's skin via the plurality of openings.

The at least one nonmetallic material may comprise nylon or cotton.

The mesh interface may provide improved skin comfort as determined by a Human Skin Irritation Test (ISO 10993-10 C3.3).

Preferred and non-limiting embodiments or aspects of the present disclosure will now be described in the following numbered clauses:

Clause 1: A support garment for use with a wearable cardiac therapeutic device, the garment comprising: a mesh interface configured to transmit therapeutic electrical pulses between at least one therapy electrode and a patient's skin, the mesh interface comprising: a plurality of dielectric fibers comprising at least one nonmetallic material; and a plurality of conductive fibers or particles interspersed with the plurality of dielectric fibers, the plurality of conductive fibers being configured to form a plurality of conductive pathways extending through the mesh interface, wherein the plurality of conductive pathways are configured to conduct the therapeutic electrical pulses through the mesh interface from the at least one therapy electrode to the patient's skin.

Clause 2: The support garment according to clause 1, further comprising at least one support pocket disposed on an inside surface of the support garment, the support pocket being configured to support the at least one therapy electrode on the support garment, wherein the mesh interface forms a part of the at least one support pocket.

Clause 3: The support garment according to clause 1 or clause 2, wherein the mesh interface comprises the plurality of dielectric fibers and the plurality of conductive fibers in a predetermined ratio configured to provide a comfortable feel on the patient's skin and to provide an electrical impedance of the plurality of conductive pathways extending through the mesh interface from the first surface to the second surface of approximately 0.01Ω-5Ω, or more particularly approximately 0.1Ω-2Ω, or more particularly approximately 0.25Ω-1.5Ω.

Clause 4: The support garment according to any one of clauses 1-3, wherein the mesh interface is configured to facilitate a transfer of conductive gel from the at least one therapy electrode to the patient's skin.

Clause 5: The support garment according to any one of clauses 1-4, wherein the mesh interface is configured to provide a comfortable feel on the patient's skin and to wick moisture away from the patient's skin.

Clause 6: The support garment according to any one of clauses 1-5, wherein the mesh interface is configured to transmit a falloff signal from the at least one therapy electrode to the patient's skin configured to determine that the at least one therapy electrode is correctly positioned on the patient's body.

Clause 7: The support garment according to any one of clauses 1-6, wherein the mesh interface is configured to provide an electrical impedance of the plurality of conductive pathways extending through the mesh interface from the first surface to the second surface of approximately 0.01Ω-5Ω, or more particularly approximately 0.1Ω-2Ω, or more particularly approximately 0.25Ω-1.5Ω.

Clause 8: The support garment according to any one of clauses 1-7, wherein the plurality of conductive fibers or particles comprises an impedance measure of approximately 10-250 Ω/meter, or more particularly approximately 20-150 Ω/meter, or more particularly approximately 30-130 Ω/meter.

Clause 9: The support garment according to any one of clauses 1-8, wherein the mesh interface further comprises a plurality of openings extending through the mesh interface from the first surface to the second surface, the mesh interface being configured to facilitate transfer of conductive gel from the at least one therapy electrode to the patient's skin via the plurality of openings.

Clause 10: The support garment according to clause 9, wherein the mesh interface is configured to receive conductive gel from a plurality of holes in the at least one therapy electrode in an amount of approximately 0.1 cubic-centimeter (cc) to 30 cc of conductive gel, or more particularly approximately 0.5 cc to 20 cc, or more particularly approximately 0.9 cc to 10 cc.

Clause 11: The support garment according to clause 10, wherein the conductive gel is configured to provide a predetermined electrical impendence of the plurality of conductive pathways extending through the mesh interface from the first surface to the second surface of approximately 0.01Ω-5Ω, or more particularly approximately 0.1Ω-2Ω, or more particularly approximately 0.25Ω-1.5Ω.

Clause 12: The support garment according to any one of clauses 1-11, wherein the mesh interface is configured to be porous to the conductive gel from a plurality of holes in the at least one therapy electrode to provide a predetermined electrical impendence of the plurality of conductive pathways extending through the mesh interface from the first surface to the second surface of approximately 0.01Ω-5Ω, or more particularly approximately 0.1Ω-2Ω, or more particularly approximately 0.25Ω-1.5Ω.

Clause 13: The support garment according to clause 12, wherein the mesh interface comprises a plurality of openings, the plurality of opening comprising approximately 2-1000 openings per square inch of the mesh interface, or more particularly approximately 5-500 openings per square inch of the mesh interface, or more particularly approximately 10-100 openings per square inch of the mesh interface.

Clause 14: The support garment according to clause 12 or clause 13, wherein the mesh interface comprises a plurality of openings having an average diameter in a range of approximately 0.005″-0.3″ (0.13 mm-7.6 mm), or more particularly approximately 0.01″-0.2″ (0.25 mm-5.1 mm), or more particularly approximately 0.05″-0.1″ (1.3 mm-2.5 mm).

Clause 15: The support garment according to any one of clauses 1-14, wherein the plurality of dielectric fibers of the mesh interface comprise a dielectric yarn and the plurality of conductive fibers or particles of the mesh interface comprise a conductive yarn, and wherein the dielectric yarn and the conductive yarn are intertwined together to form the mesh interface.

Clause 16: The support garment according to clause 15, wherein the conductive yarn comprises silver-plated nylon yarn, and/or nickel-plated or metalized yarn, and/or aluminum-plated or metalized yarn, and/or carbon coated yarn, and/or carbon filled yarn.

Clause 17: The support garment according to any one of clauses 1-16, wherein the at least one nonmetallic material comprises nylon or cotton.

Clause 18: The support garment according to any one of clauses 1-17, wherein the mesh interface provides improved skin comfort as determined by a Human Skin Irritation Test (ISO 10993-10 C3.3).

Clause 19: A wearable cardiac therapeutic device for improved skin comfort when worn by a patient, the device comprising: at least one therapy electrode configured to deliver therapeutic electrical pulses to a patient's heart; and a support garment configured to support the at least one therapy electrode in electrical communication with the patient's body, the support garment comprising: at least one support pocket disposed on an inside surface of the support garment for supporting the at least one therapy electrode on the support garment; and a mesh interface formed as part of the at least one support pocket, the mesh interface configured to facilitate electrical contact between the at least one therapy electrode and the patient's skin, wherein the mesh interface comprises: a first surface oriented toward the at least one therapy electrode; a second surface oriented toward the patient's skin; a plurality of dielectric fibers comprising at least one nonmetallic material; and a plurality of conductive fibers or particles, wherein the plurality of dielectric fibers and the plurality of conductive fibers or particles are interspersed to form a plurality of conductive pathways extending through the mesh interface from the first surface to the second surface, the plurality of conductive pathways being configured to conduct the therapeutic electrical pulses through the mesh interface from the at least one therapy electrode to the patient.

Clause 20: The wearable cardiac therapeutic device according to clause 19, wherein the mesh interface is configured to facilitate a transfer of conductive gel from the at least one therapy electrode to the patient's skin.

Clause 21: The wearable cardiac therapeutic device according to clause 19 or clause 20, wherein the mesh interface is configured to provide a comfortable feel on the patient's skin and to wick moisture away from the patient's skin.

Clause 22: The wearable cardiac therapeutic device according to any one of clauses 19-21, wherein the mesh interface is configured to transmit a falloff signal from the at least one therapy electrode to the patient's skin configured to determine that the at least one therapy electrode is correctly positioned on the patient's body.

Clause 23: The wearable cardiac therapeutic device according to any one of clauses 19-22, wherein the mesh interface is configured to provide an electrical impedance of the plurality of conductive pathways extending through the mesh interface from the first surface to the second surface of approximately 0.01Ω-5Ω, or more particularly approximately 0.1Ω-2Ω, and or more particularly approximately 0.25Ω-1.5Ω.

Clause 24: The wearable cardiac therapeutic device according to any one of clauses 19-23, wherein the plurality of conductive fibers or particles comprises an impedance measure of approximately 10-250 Ω/meter, or more particularly approximately 20-150 Ω/meter, and or more particularly approximately 30-130 Ω/meter.

Clause 25: The wearable cardiac therapeutic device according to any one of clauses 19-24, wherein the mesh interface further comprises a plurality of openings extending through the mesh interface from the first surface to the second surface, the mesh interface being configured to facilitate transfer of conductive gel from the at least one therapy electrode to the patient's skin via the plurality of openings.

Clause 26: The wearable cardiac therapeutic device according to clause 25, wherein the mesh interface is configured to receive conductive gel from a plurality of holes in the at least one therapy electrode in an amount of approximately 0.1 cubic-centimeter (cc) to 30 cc of conductive gel, or more particularly approximately 0.5 cc to 20 cc, and or more particularly approximately 0.9 cc to 10 cc.

Clause 27: The wearable cardiac therapeutic device according to clause 26, wherein the conductive gel is configured to provide a predetermined electrical impendence of the plurality of conductive pathways extending through the mesh interface from the first surface to the second surface of approximately 0.01Ω-5Ω, or more particularly approximately 0.1Ω-2Ω, and or more particularly approximately 0.25Ω-1.5Ω.

Clause 28: The wearable cardiac therapeutic device according to any one of clauses 19-27, wherein the mesh interface is configured to be porous to conductive gel from a plurality of holes in the at least one therapy electrode to provide a predetermined electrical impendence of the plurality of conductive pathways extending through the mesh interface from the first surface to the second surface of approximately 0.01Ω-5Ω, or more particularly approximately 0.1Ω-2Ω, and or more particularly approximately 0.25Ω-1.5Ω.

Clause 29: The wearable cardiac therapeutic device according to clause 28, wherein the mesh interface comprises a plurality of openings, the plurality of opening comprising approximately 2-1000 openings per square inch of the mesh interface, or more particularly approximately 5-500 openings per square inch of the mesh interface, and or more particularly approximately 10-100 openings per square inch of the mesh interface.

Clause 30: The wearable cardiac therapeutic device according to clause 28 or clause 29, wherein the mesh interface comprises a plurality of openings having an average diameter in a range of approximately 0.005″-0.3″ (0.13 mm-7.6 mm), or more particularly approximately 0.01″-0.2″ (0.25 mm-5.1 mm), and or more particularly approximately 0.05″-0.1″ (1.3 mm-2.5 mm).

Clause 31: The wearable cardiac therapeutic device according to any one of clauses 19-30, wherein a thickness of the mesh interface from the first surface to the second surface is approximately 0.005″-0.5″ (0.13 mm-12.7 mm), or more particularly approximately 0.01″-0.25″ (0.25 mm-6.4 mm), and or more particularly approximately 0.03″-0.1″ (0.76 mm-2.5 mm).

Clause 32: The wearable cardiac therapeutic device according to any one of clauses 19-31, wherein the dielectric fibers comprise, at least in part, fusible fibers that are configured to shrink in volume relative to the conductive fibers or particles when the mesh interface is exposed to heat.

Clause 33: The wearable cardiac therapeutic device according to clause 32, wherein the fusible fibers are configured to shrink in volume relative to the conductive fibers or particles when the mesh interface is exposed to heat resulting in the conductive fibers or particles expressing more relative to the dielectric fibers, whereby the plurality of conductive pathways extending through the mesh interface project from the first surface and the second surface of the mesh interface.

Clause 34: The wearable cardiac therapeutic device according to any one of clauses 19-33, wherein the plurality of dielectric fibers of the mesh interface comprise a dielectric yarn and the plurality of conductive fibers or particles of the mesh interface comprise a conductive yarn, and wherein the dielectric yarn and the conductive yarn are intertwined together to form the mesh interface.

Clause 35: The wearable cardiac therapeutic device according to clause 34, wherein the dielectric yarn comprises a textured nylon or cotton yarn and a fusible yarn.

Clause 36: The wearable cardiac therapeutic device according to clause 34 or clause 35, wherein the conductive yarn comprises silver-plated nylon yarn, and/or nickel-plated or metalized yarn, and/or aluminum-plated or metalized yarn, and/or carbon coated yarn, and/or carbon filled yarn.

Clause 37: The wearable cardiac therapeutic device according to any one of clauses 34-36, wherein the mesh interface comprises approximately 10%-60% by weight of conductive yarn, or more particularly approximately 15%-40% by weight of conductive yarn, and or more particularly approximately 20%-35% by weight of conductive yarn.

Clause 38: The wearable cardiac therapeutic device according to any one of clauses 34-37, wherein the mesh interface comprises a plurality of intertwined structures of the dielectric yarn and the conductive yarn.

Clause 39: The wearable cardiac therapeutic device according to clause 38, wherein the plurality of intertwined structures comprises a pattern of at least three intertwined structures.

Clause 40: The wearable cardiac therapeutic device according to clause 39, wherein the at least three intertwined structures comprise: a plurality of courses of the dielectric yarn intertwined with each other in a tubular pattern structure; at least one course of the conductive yarn intertwined with the plurality of courses of the dielectric yarn in a 1×1 rib pattern structure; and at least one pointelle pattern structure of intertwined dielectric yarn.

Clause 41: The wearable cardiac therapeutic device according to clause 40, wherein the dielectric yarn comprises a textured nylon or cotton yarn and a fusible yarn arranged together in a plated yarn structure, and wherein the textured nylon or cotton yarn forms an exterior of the plurality of courses of the tubular pattern structure and the fusible yarn forms an interior of the plurality of courses of the tubular pattern structure.

Clause 42: The wearable cardiac therapeutic device according to clause 41, wherein the at least one course of the conductive yarn in the 1×1 rib pattern structure extends from the first surface of the mesh interface to the second surface of the mesh interface.

Clause 43: The wearable cardiac therapeutic device according to clause 42, wherein the at least one 1×1 rib pattern structure is configured such that the conductive yarn stands out of the first and second surfaces of the mesh interface.

Clause 44: The wearable cardiac therapeutic device according to clause 43, wherein the fusible yarn is configured to melt, dissipate, and/or shrink in volume relative to the conductive yarn when exposed to heat to cause the dielectric yarn to contract relative to the conductive yarn and enhance the standing out of the conductive yarn from the first and second surfaces of the mesh interface.

Clause 45: The wearable cardiac therapeutic device according to any one of clauses 40-44, wherein the at least one pointelle pattern structure defines a plurality of openings extending through the mesh interface from the first surface to the second surface, the mesh interface being configured to facilitate transfer of conductive gel from the at least one therapy electrode to the patient's skin via the plurality of openings.

Clause 46: The wearable cardiac therapeutic device according to any one of clauses 19-45, wherein the at least one nonmetallic material comprises nylon or cotton.

Clause 47: The wearable cardiac therapeutic device according to any one of clauses 19-46, wherein the mesh interface provides improved skin comfort as determined by a Human Skin Irritation Test (ISO 10993-10 C3.3).

Clause 48: A wearable cardiac therapeutic device for improved skin comfort when worn by a patient, the device comprising: at least one therapy electrode configured to deliver therapeutic electrical pulses to a patient's heart; and a support garment configured to support the at least one therapy electrode in electrical communication with the patient's body, the support garment comprising: at least one support pocket disposed on an inside surface of the support garment for supporting the at least one therapy electrode on the support garment; and a mesh interface formed as part of the at least one support pocket, the mesh interface configured to be facilitate electrical contact between the at least one therapy electrode and the patient's skin, wherein the mesh interface comprises: a first surface oriented toward the at least one therapy electrode; a second surface oriented toward the patient's skin; a plurality of dielectric fibers comprising at least one nonmetallic material; a plurality of conductive fibers or particles; and a plurality of openings extending through the mesh interface from the first surface to the second surface, and wherein the mesh interface is configured to facilitate a transfer of conductive gel from the at least one therapy electrode to the patient's skin via the plurality of openings.

Clause 49: The wearable cardiac therapeutic device according to clause 48, wherein the mesh interface is configured to provide a comfortable feel on the patient's skin and to wick moisture away from the patient's skin.

Clause 50: The wearable cardiac therapeutic device according to clause 48 or clause 49, wherein the mesh interface is configured to transmit a falloff signal from the at least one therapy electrode to the patient's skin configured to determine that the at least one therapy electrode is correctly positioned on the patient's body.

Clause 51: The wearable cardiac therapeutic device according to any one of clauses 48-50, wherein the plurality of dielectric fibers and the plurality of conductive fibers or particles are interspersed to form a plurality of conductive pathways extending through the mesh interface from the first surface to the second surface, the plurality of conductive pathways being configured to conduct the therapeutic electrical pulses through the mesh interface from the at least one therapy electrode to the patient.

Clause 52: The wearable cardiac therapeutic device according to clause 51, wherein the mesh interface is configured to provide an electrical impedance of the plurality of conductive pathways extending through the mesh interface from the first surface to the second surface of approximately 0.01Ω-5Ω, or more particularly approximately 0.1Ω-2Ω, and or more particularly approximately 0.25Ω-1.5Ω.

Clause 53: The wearable cardiac therapeutic device according to clause 51 or clause 52, wherein the plurality of conductive fibers or particles comprises an impedance measure of approximately 10-250 Ω/meter, or more particularly approximately 20-150 Ω/meter, and or more particularly approximately 30-130 Ω/meter.

Clause 54: The wearable cardiac therapeutic device according to any one of clauses 51-53, wherein the mesh interface is configured to receive conductive gel from a plurality of holes in the at least one therapy electrode in an amount of approximately 0.1 cubic-centimeter (cc) to 30 cc of conductive gel, or more particularly approximately 0.5 cc to 20 cc, and or more particularly approximately 0.9 cc to 10 cc.

Clause 55: The wearable cardiac therapeutic device according to clause 54, wherein the conductive gel is configured to provide a predetermined electrical impendence of the plurality of conductive pathways extending through the mesh interface from the first surface to the second surface of approximately 0.01Ω-5Ω, or more particularly approximately 0.1Ω-2Ω, and or more particularly approximately 0.25Ω-1.5Ω.

Clause 56: The wearable cardiac therapeutic device according to any one of clauses 51-55, wherein the mesh interface is configured to be porous to the conductive gel from a plurality of holes in the at least one therapy electrode to provide a predetermined electrical impendence of the plurality of conductive pathways extending through the mesh interface from the first surface to the second surface of approximately 0.01Ω-5Ω, or more particularly approximately 0.1Ω-2Ω, and or more particularly approximately 0.25Ω-1.5Ω.

Clause 57: The wearable cardiac therapeutic device according to clause 56, wherein the plurality of openings comprises approximately 2-1000 openings per square inch of the mesh interface, or more particularly approximately 5-500 openings per square inch of the mesh interface, and or more particularly approximately 10-100 openings per square inch of the mesh interface.

Clause 58: The wearable cardiac therapeutic device according to clause 56 or clause 57, wherein the plurality of openings have an average diameter in a range of approximately 0.005″-0.3″ (0.13 mm-7.6 mm), or more particularly approximately 0.01″-0.2″ (0.25 mm-5.1 mm), and or more particularly approximately 0.05″-0.1″ (1.3 mm-2.5 mm).

Clause 59: The wearable cardiac therapeutic device according to any one of clauses 48-58, wherein a thickness of the mesh interface from the first surface to the second surface is approximately 0.005″-0.5″ (0.13 mm-12.7 mm), or more particularly approximately 0.01″-0.25″ (0.25 mm-6.4 mm), and or more particularly approximately 0.03″-0.1″ (0.76 mm-2.5 mm).

Clause 60: The wearable cardiac therapeutic device according to any one of clauses 48-59, wherein the dielectric fibers comprise, at least in part, fusible fibers that are configured to shrink in volume relative to the conductive fibers or particles when the mesh interface is exposed to heat.

Clause 61: The wearable cardiac therapeutic device according to claim 60, wherein the fusible fibers are configured to shrink in volume relative to the conductive fibers or particles when the mesh interface is exposed to heat resulting the conductive fibers or particles expressing more relative to the dielectric fibers, whereby the plurality of conductive pathways extending through the mesh interface project from the first surface and the second surface of the mesh interface.

Clause 62: The wearable cardiac therapeutic device according to any one of clauses 48-61, wherein the plurality of dielectric fibers of the mesh interface comprise a dielectric yarn and the plurality of conductive fibers or particles of the mesh interface comprise a conductive yarn, and wherein the dielectric yarn and the conductive yarn are intertwined together to form the mesh interface.

Clause 63: The wearable cardiac therapeutic device according to clause 62, wherein the dielectric yarn comprises a textured nylon or cotton yarn and a fusible yarn.

Clause 64: The wearable cardiac therapeutic device according to clause 62 or clause 63, wherein the conductive yarn comprises silver-plated nylon yarn, and/or nickel-plated or metalized yarn, and/or aluminum-plated or metalized yarn, and/or carbon coated yarn, and/or carbon filled yarn.

Clause 65: The wearable cardiac therapeutic device according to any one of clauses 62-64, wherein the mesh interface comprises approximately 10%-60% by weight of conductive yarn, or more particularly approximately 15%-40% by weight of conductive yarn, and or more particularly approximately 20%-35% by weight of conductive yarn.

Clause 66: The wearable cardiac therapeutic device according to any one of clauses 62-65, wherein the mesh interface comprises a plurality of intertwined structures of the dielectric yarn and the conductive yarn.

Clause 67: The wearable cardiac therapeutic device according to clause 66, wherein the plurality of intertwined structures comprises a pattern of at least three intertwined structures.

Clause 68: The wearable cardiac therapeutic device according to clause 67, wherein the at least three intertwined structures comprise: a plurality of courses of the dielectric yarn intertwined with each other in a tubular pattern structure; at least one course of the conductive yarn intertwined with the plurality of courses of the dielectric yarn in a 1×1 rib pattern structure; and at least one pointelle pattern structure of intertwined dielectric yarn.

Clause 69: The wearable cardiac therapeutic device according to clause 68, wherein the dielectric yarn comprises a textured nylon or cotton yarn and a fusible yarn arranged together in a plated yarn structure, and wherein the textured nylon or cotton yarn forms an exterior of the plurality of courses of the tubular pattern structure and the fusible yarn forms an interior of the plurality of courses of the tubular pattern structure.

Clause 70: The wearable cardiac therapeutic device according to clause 69, wherein the at least one course of the conductive yarn in the 1×1 rib pattern structure extends from the first surface of the mesh interface to the second surface of the mesh interface.

Clause 71: The wearable cardiac therapeutic device according to clause 70, wherein the at least one 1×1 rib pattern structure is configured such that the conductive yarn stands out of the first and second surfaces of the mesh interface.

Clause 72: The wearable cardiac therapeutic device according to clause 71, wherein the fusible yarn is configured to melt, dissipate, and/or shrink in volume relative to the conductive yarn when exposed to heat to cause the dielectric yarn to contract relative to the conductive yarn and enhance the standing out of the conductive yarn from the first and second surfaces of the mesh interface.

Clause 73: The wearable cardiac therapeutic device according to any one of clauses 68-72, wherein the at least one pointelle pattern structure defines a plurality of openings extending through the mesh interface from the first surface to the second surface, the mesh interface being configured to facilitate transfer of conductive gel from the at least one therapy electrode to the patient's skin via the plurality of openings.

Clause 74: The wearable cardiac therapeutic device according to any one of clauses 48-73, wherein the at least one nonmetallic material comprises nylon or cotton.

Clause 75: The wearable cardiac therapeutic device according to any one of clauses 48-74, wherein the mesh interface provides improved skin comfort as determined by a Human Skin Irritation Test (ISO 10993-10 C3.3).

### BRIEF DESCRIPTION OF THE DRAWINGS

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limit of the invention.

Further features and other examples and advantages will become apparent from the following detailed description made with reference to the drawings.

FIG. 1 is a schematic of an example wearable cardiac monitoring and therapeutic medical device that may be used in connection with the present disclosure.

FIG. 2 is a front view of an example support garment for the wearable cardiac monitoring and therapeutic medical device of FIG. 1 as worn on a patient.

FIG. 3 is a rear view of the support garment of FIG. 2 as worn on a patient.

FIGS. 4A and 4B are a front view of an example support garment and electrode assembly for the wearable monitoring and therapeutic medical device that may be used in connection with the present disclosure.

FIG. 5 is an enlarged front view of a mesh interface for a support pocket on the support garment of FIGS. 4A and 4B that may be used in connection with the present disclosure.

FIG. 6 is a further enlarged view of the mesh interface of FIG. 5 taken from area VI shown in FIG. 5.

FIG. 6A is a photograph of a sample mesh interface according to the view of FIG. 6.

FIG. 7 is a schematic perspective illustration of a portion of the mesh interface of FIG. 5.

FIG. 8 is a schematic cross-sectional illustration of the mesh interface taken along lines 8-8 shown in FIG. 7 as applied between a therapy electrode and a patient's skin.

FIGS. 9A and 9B are rear and front views, respectively, of a mannequin testing arrangement for measuring an impedance of the mesh interface according to an example of the present disclosure.

FIG. 10 is a schematic of a pattern of intertwined yarn structures forming the mesh interface of FIG. 5 according to an example the present disclosure.

FIG. 10A is an illustration of the schematic of FIG. 10.

FIGS. 11A and 11B are enlarged front views of the mesh interface of FIG. 5 prior to and after an applied heat exposure.

FIG. 12 is a schematic of an example of a wearable cardiac monitoring and therapeutic medical device that may be used in connection with the present disclosure.

FIG. 13A is a schematic drawing showing a front perspective view of an example monitor for the wearable medical device of FIG. 12.

FIG. 13B is a schematic drawing showing a rear perspective view of the example monitor of FIG. 13A.

FIG. 14 is a schematic diagram of functional components of the wearable medical device of FIG. 12.

FIG. 15 is a photographic top view of a sample mesh interface prior to stretching and an applied heat exposure according to an example of the present disclosure.

FIG. 16 is a photographic top view of the sample mesh interface of FIG. 15 after stretching, but prior to an applied heat exposure.

FIG. 17 is an enlarged photographic view of a portion of the sample mesh interface of FIG. 15.

FIG. 18 is an enlarged cross-sectional photographic view of a portion of the sample mesh interface of FIG. 15.

FIG. 18A is an enlarged photographic top view of a portion of the sample mesh interface of FIG. 15.

FIG. 19 is a photographic top view of the sample mesh interface of FIG. 15 after stretching and an applied heat exposure.

FIG. 20 is an enlarged photographic view of a portion of the sample mesh interface of FIG. 19.

FIG. 21 is a photographic side view of the sample mesh interface of FIG. 19.

FIG. 22 is an enlarged photographic view of a portion of the sample mesh interface of FIG. 21.

FIG. 23 is another photographic side view of the sample mesh interface of FIG. 19.

FIG. 24 is an enlarged photographic view of a portion of the sample mesh interface of FIG. 23.

FIG. 25 is another photographic side view of the sample mesh interface of FIG. 19.

FIG. 26 is an enlarged photographic view of a portion of the sample mesh interface of FIG. 25.

FIG. 27 is another photographic side view of the sample mesh interface of FIG. 19.

FIG. 28 is an enlarged photographic view of a portion of the sample mesh interface of FIG. 27.

FIG. 29 is another photographic side view of the sample mesh interface of FIG. 19.

FIG. 30 is an enlarged photographic view of a portion of the sample mesh interface of FIG. 29.

### DETAILED DESCRIPTION OF SOME OF THE EMBODIMENTS

As used herein, the singular forms of “a”, “an”, and “the” include plural referents unless the context clearly dictates otherwise.

As used herein, the terms “right”, “left”, “top”, and derivatives thereof shall relate to the invention as it is oriented in the drawing figures. However, it is to be understood that the invention can assume various alternative orientations and, accordingly, such terms are not to be considered as limiting. Also, it is to be understood that the invention can assume various alternative variations and stage sequences, except where expressly specified to the contrary. It is also to be understood that the specific devices and processes illustrated in the attached drawings and described in the following specification are examples. Hence, specific dimensions and other physical characteristics related to the embodiments disclosed herein are not to be considered as limiting.

For the purposes of this specification, unless otherwise indicated, all numbers expressing quantities of ingredients, reaction conditions, dimensions, physical characteristics, and so forth used in the specification and claims are to be understood as being modified in all instances by the term “about” or “approximately”. Unless indicated to the contrary, the numerical parameters set forth in the following specification and attached claims are approximations that can vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements. Also, it should be understood that any numerical range recited herein is intended to include all subranges subsumed therein. For example, a range of “1 to 10” is intended to include any and all subranges between and including the recited minimum value of 1 and the recited maximum value of 10; that is, all subranges beginning with a minimum value equal to or greater than 1 and ending with a maximum value equal to or less than 10, and all subranges in between, e.g., 1 to 6.3, or 5.5 to 10, or 2.7 to 6.1.

As used herein, the terms “communication” and “communicate” refer to the receipt or transfer of one or more signals, messages, commands, or other type of data. For one unit or component to be in communication with another unit or component means that the one unit or component is able to directly or indirectly receive data from and/or transmit data to the other unit or component. This can refer to a direct or indirect connection that can be wired and/or wireless in nature. Additionally, two units or components can be in communication with each other even though the data transmitted can be modified, processed, routed, and the like, between the first and second unit or component. For example, a first unit can be in communication with a second unit even though the first unit passively receives data and does not actively transmit data to the second unit. As another example, a first unit can be in communication with a second unit if an intermediary unit processes data from one unit and transmits processed data to the second unit. It will be appreciated that numerous other arrangements are possible.

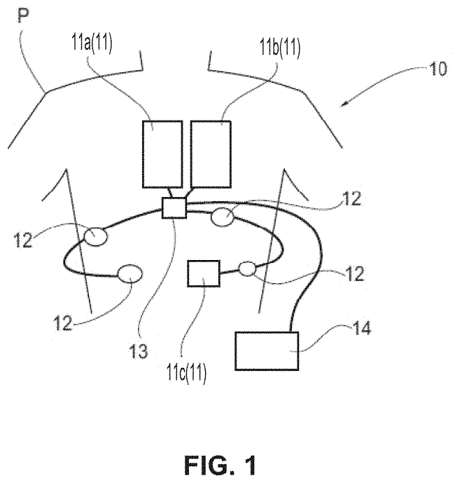

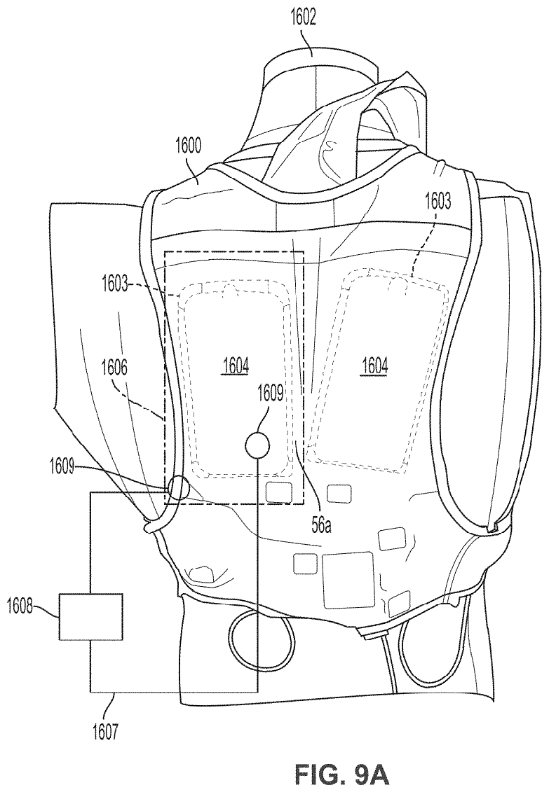

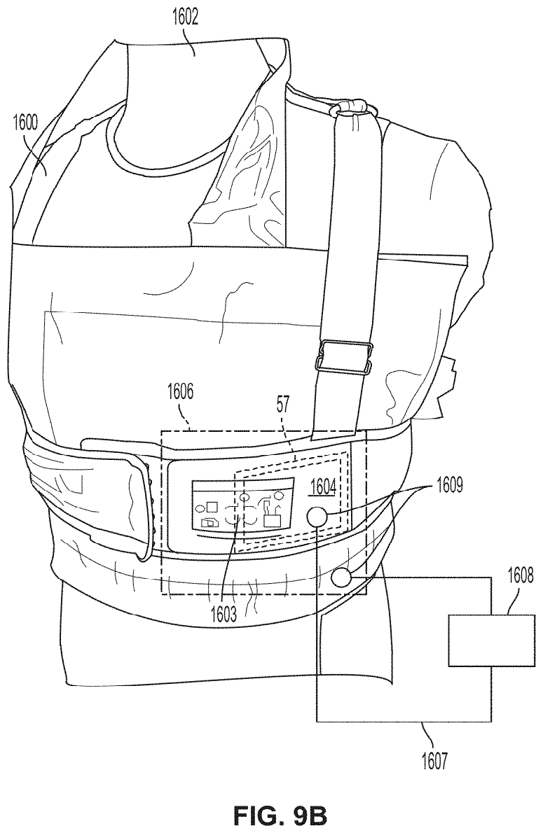

FIG. 1 illustrates an example wearable medical device 10 that is external, ambulatory, and wearable by a patient P and is configured to implement one or more configurations described herein. For example, the wearable medical device 10 can be an external or non-invasive medical device, e.g., the device 10 configured to be located substantially external to the patient P. For example, the wearable medical device 10, shown in FIG. 1 as a wearable defibrillator 10, as described herein can be bodily-attached to the patient such as the LifeVest® wearable cardioverter defibrillator available from ZOLL® Medical Corporation of Pittsburgh, Pa. and Chelmsford, Mass. The wearable defibrillator 10 can be worn or carried by an ambulatory patient P. According to one example of the present disclosure, the wearable defibrillator 10 is used as an ambulatory cardiac monitoring and treatment device within a monitoring and treatment system according to the present disclosure. FIGS. 9-11, discussed in detail below, illustrate in further detail an example wearable medical device 100 that may be used in connection with the present disclosure.

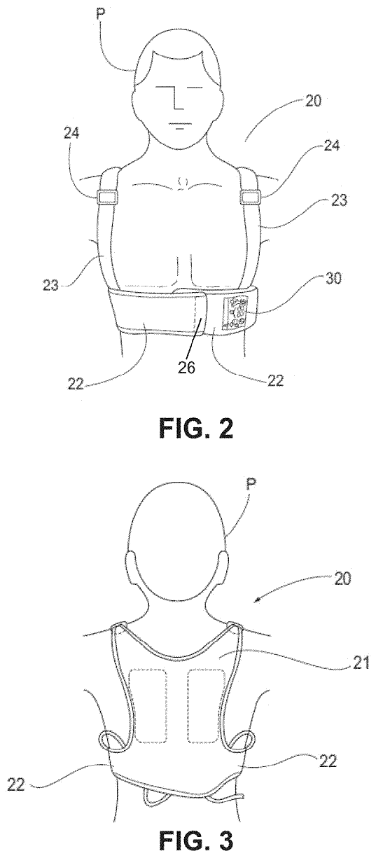

In accordance with one or more examples, a support garment 20 is provided to keep the electrodes 11 and sensing electrodes 12 in place against the patient's body while remaining comfortable during wear. FIGS. 2 and 3 illustrate such a support garment 20 in accordance with an example of the present disclosure. Such an example support garment is described in U.S. Pat. No. 9,782,578 titled “Patient-worn Energy Delivery Apparatus and Techniques for Sizing Same”, the content of which is hereby incorporated by reference.

In order to obtain a reliable ECG signal so that the monitor can function effectively and reliably, the sensing electrodes 12 must be in the proper position and in good contact with the patient's skin. The electrodes 12 need to remain in a substantially fixed position and not move excessively or lift off the skin's surface. If there is excessive movement or lifting, the ECG signal will be adversely affected with noise and can cause problems with the arrhythmia detection and in the ECG analysis and monitoring system. Similarly, in order to effectively deliver the defibrillating energy, the therapy electrodes, e.g., two rear therapy electrodes 11a and 11b, and a front therapy electrode 11c (collectively therapy electrodes 11) must be in the proper position and in good contact with the patient's skin. If the therapy electrodes 11 are not firmly positioned against the skin, there can be problems with high impedance, leading to a less effective delivery of energy. If the therapy electrodes 11 are not firmly positioned, there can also be damage to the patient's skin, such as burning, when the shock is delivered.

In accordance with one or more examples, the support garment 20 may provide comfort and functionality under circumstances of human body dynamics, such as bending, twisting, rotation of the upper thorax, semi-reclining, and lying down. These are also positions that a patient may assume if he/she were to become unconscious due to an arrhythmic episode. The design of the garment 20 is generally such that it minimizes bulk, weight, and undesired concentrations of force or pressure while providing the necessary radial forces upon the treatment and sensing electrodes 11, 12 to ensure device functionality. A wearable defibrillator monitor 14 may be disposed in a support holster (not shown) operatively connected to or separate from the support garment 20. The support holster may be incorporated in a band or belt worn about the patient's waist or thigh.

As shown in FIGS. 2 and 3, the support garment 20 may be provided in the form of a vest or harness having a back portion 21 and sides extending around the front of the patient P to form a belt 22. The ends of the belt 22 are connected at the front of the patient P by a closure 26, which may comprise one or more clasps. Multiple corresponding closures may be provided along the length of the belt 22 to allow for adjustment in the size of the secured belt 22 in order to provide a more customized fit to the patient P. The support garment 20 may further include two straps 23 connecting the back portion 21 to the belt 22 at the front of the patient P. The straps 23 have an adjustable size to provide a more customized fit to the patient P. The straps 23 may be provided with sliders 24 to allow for the size adjustment of the straps 23. The straps 23 may also be selectively attached to the belt 22 at the front of the patient P. The support garment 20 may be comprised of an elastic, low spring rate material that stretches appropriately to keep the electrodes 11, 12 in place against the patient's skin while the patient P moves and is lightweight and breathable. For example, the support garment 20 may have elastic, low spring rate material composition based on a fiber content of about 20% elastic fiber, 32% polyester fiber, and up to 48% or more of nylon or other fiber. Appropriate materials for the support garment 20 are discussed in detail in the above-mentioned U.S. Pat. No. 9,782,578.

In accordance with one or more examples, the support garment 20 may be formed from an elastic, low spring rate material and constructed using tolerances that are considerably closer than those customarily used in garments. The materials for construction are chosen for functionality, comfort, and biocompatibility. The materials may be configured to wick perspiration from the skin. The support garment 20 may be formed from one or more blends of nylon, polyester, and spandex fabric material. Different portions or components of the support garment 20 may be formed from different material blends depending on the desired flexibility and stretchability of the support garment 20 and/or its specific portions or components. For instance, the belt 22 of the support garment 20 may be formed to be more stretchable than the back portion 21. According to one example, the support garment 20 is formed from a blend of nylon and spandex materials, such as a blend of 77% nylon and 23% spandex. According to another example, the support garment 20 is formed from a blend of nylon, polyester, and spandex materials, such as 40% nylon, 32% polyester, and 14% spandex. According to another example, the support garment 20 is formed from a blend of polyester and spandex materials, such as 86% polyester and 14% spandex or 80% polyester and 20% spandex. For example, the nylon and spandex material is configured to be aesthetically appealing, and comfortable, e.g., when in contact with the patient's skin. Stitching within the support garment 20 may be made with industrial stitching thread. According to one example, the stitching within the support garment 20 is formed from a cotton-wrapped polyester core thread.

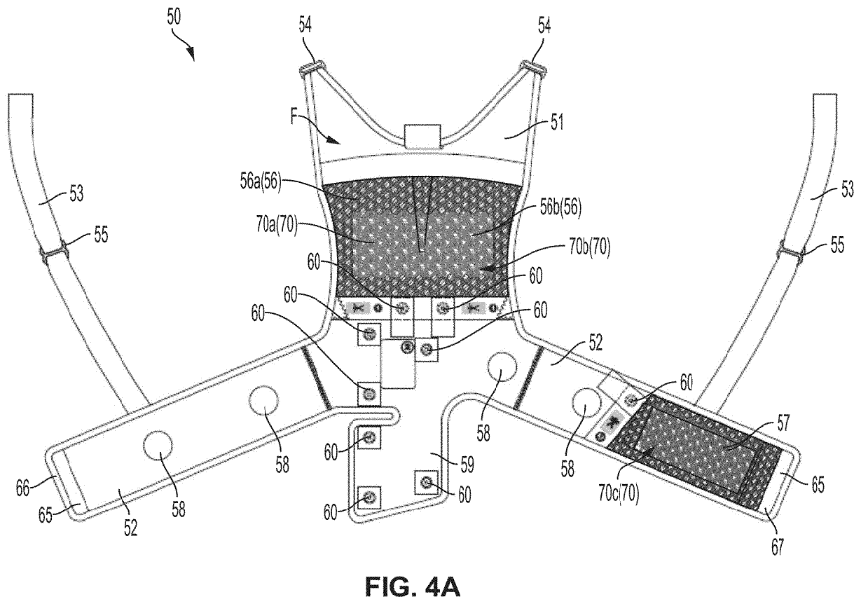

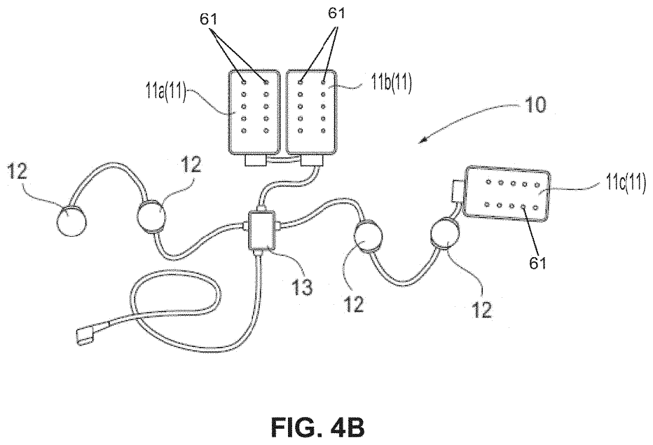

FIGS. 4A-11B illustrate an example support garment 50 according to the present disclosure. The support garment 50 incorporates additional improvements for enhancing the patient's experience in wearing the support garment for an extended period of time. The support garment examples provided herein promote comfort, aesthetic appearance, and ease of use or application for older patients, or patients with physical infirmities and/or who are physically challenged, including patients with rheumatic conditions, patients with arthritis, and/or patients with autoimmune or inflammatory diseases that affect joints, tendons, ligaments, bones, and muscles of the arm and hand. Patients afflicted with such conditions can properly and/or correctly don the garments described herein. Features of the support garments may also help minimize the time needed by patients to assemble, don or remove the support garment. Further, patients benefit from such features, which can facilitate longer wear times, better patient compliance, and improve the reliability of the detected physiological signals and treatment of the patient. These features promote ease of use, comfort and an aesthetic appearance for such patient populations. For example, the features include support pockets for the therapeutic electrodes 11 that incorporate rear pocket mesh interfaces 70a and 70b, and a front pocket mesh interface 70c (collectively mesh interface 70) between the therapeutic electrodes 11 and the patient's skin that is more comfortable, less abrasive, and less likely to cause irritation to the patient's skin or a negative reaction. The patient may be required to wear the support garment and the components continuously or nearly continuously for extensive periods of time. Over these extensive periods of time, the patient may experience discomfort while wearing the support garment as a result of the abrasiveness of the metal materials contained within the interfacing fabric material. The patient may also develop a negative reaction to the metal material over time. Patients may benefit from a wearable cardioverter defibrillator garment that includes features for enhancing the patient's experience in wearing the support garment with respect to wearability and comfort of the garment with respect to the interfacing fabric materials.

These features can encourage patients to wear the support garment and associated medical device for longer and/or continuous periods of time with minimal interruptions in the periods of wear. For example, by minimizing interruptions in periods of wear and/or promoting longer wear durations, patients and caregivers can be assured that the device is providing desirable information about as well as protection from adverse cardiac events such as ventricular tachycardia and/or ventricular fibrillation, among others. Moreover, when the patient's wear time and/or compliance is improved, the device can collect information on arrhythmias that are not immediately life-threatening, but may be useful to monitor for the patient's cardiac health. Such arrhythmic conditions can include onset and/or offset of bradycardia, tachycardia, atrial fibrillation, pauses, ectopic beats bigeminy, trigeminy events among others. For instance, episodes of bradycardia, tachycardia, or atrial fibrillation can last several minutes and/or hours. The support garments herein provide features that encourage patients to keep the device on for longer and/or uninterrupted periods of time, thereby increasing the quality of data collected about such arrhythmias. Additionally, features as described herein, including, the mesh interfaces for the therapeutic electrode support pockets promotes better patient compliance resulting in lower false positives and noise in the physiological signals collected from ECG electrodes and other sensors disposed within the support garment. For example, when patients wear the device for longer and/or uninterrupted periods of time, the device tracks cardiac events and distinguishes such events from noise over time.

The improvements incorporated in the support garment 50 may provide comfort and wearability to the patient by utilizing a mesh interface 70 made from a layer or layers of fabric material incorporating a reduced amount of conductive metal content. The fabric material of the mesh interface may incorporate component materials that have a soft, comfortable feel on the patient's skin and are configured to wick moisture away from the patient's skin. The fabric material of the mesh interface may be less abrasive to the patient's skin and less likely to cause irritation to the patient's skin or a negative reaction.

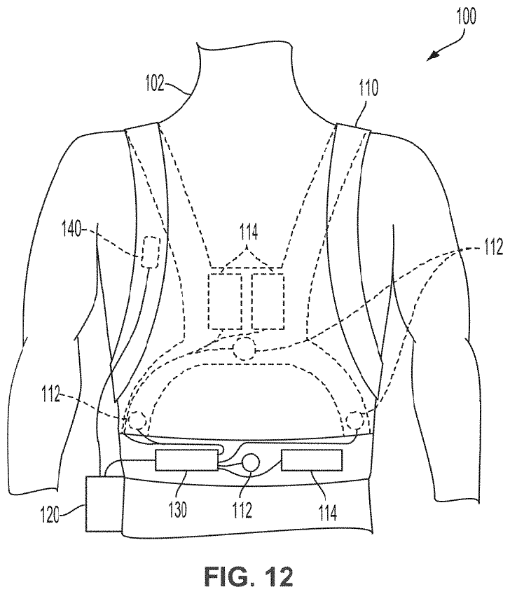

In accordance with one or more examples, the support garment 50 is provided to keep the electrodes 11, 12 of an electrode assembly 25 associated with a wearable cardiac therapeutic device in place against the patient's body while remaining comfortable to wear. In particular, the electrode assembly 25 may include a plurality of ECG sensing electrodes 12 configured to sense ECG signals regarding a cardiac function of the patient and a plurality of therapy electrodes 11 configured to deliver transcutaneous defibrillation shocks or transcutaneous pacing pulses or other types of therapeutic electrical pulses, to the patient's heart. Examples of the wearable cardiac therapeutic devices in which the support garment 50 may be utilized include the wearable medical device 14 described above with reference to FIG. 1 and the wearable medical device 100 described in detail below with reference to FIGS. 12-14. It is to be appreciated that the support garment 50 described herein may be utilized in connection with a wearable medical device of any suitable type or configuration.

As shown in FIGS. 4A and 4B, the support garment 50 may be provided in the form of a vest or harness having a back portion 51 and sides extending around the front of the patient to form a belt 52. The ends 66, 67 of the belt 52 are connected at the front of the patient by a closure mechanism 65. The support garment 50 may further include two straps 53 connecting the back portion 51 to the belt 52 at the front of the patient. The straps 53 have an adjustable size to provide a more customized fit to the patient. First strap slides 54 may be provided to connect the straps 53 to the back portion 51 of the support garment. Second strap slides 55 may be provided along the straps 53 to facilitate size adjustment of the straps 53. The straps 53 may also be selectively attached to the belt 22 at the front of the patient. The support garment 50 may be comprised of an elastic, low spring rate fabric material F that stretches appropriately to keep the electrodes 11, 12 in place against the patient's skin and is lightweight and breathable. The component materials of the fabric material F may be chosen for functionality, comfort, and biocompatibility. The component materials may be configured to wick perspiration from the skin. For example, the fabric material F may comprise a tricot fabric, the tricot fabric comprising nylon and spandex materials. The tricot fabric may comprise approximately 65%-90% nylon material, or more particularly 70%-85% nylon material, or more particularly 77% nylon material. It is to be appreciated that the fabric material F chosen for the support garment 50 may be comprised of any suitable materials or combinations of materials.

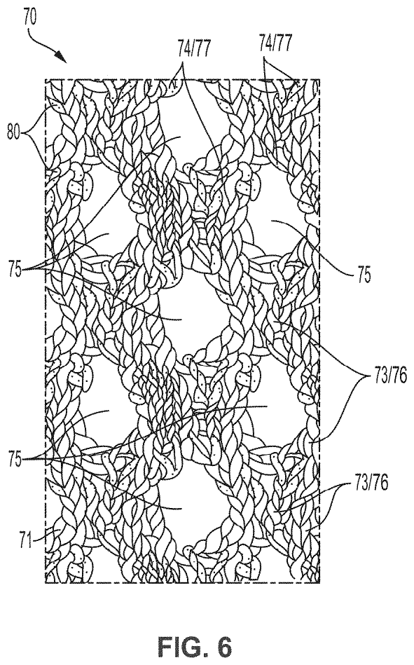

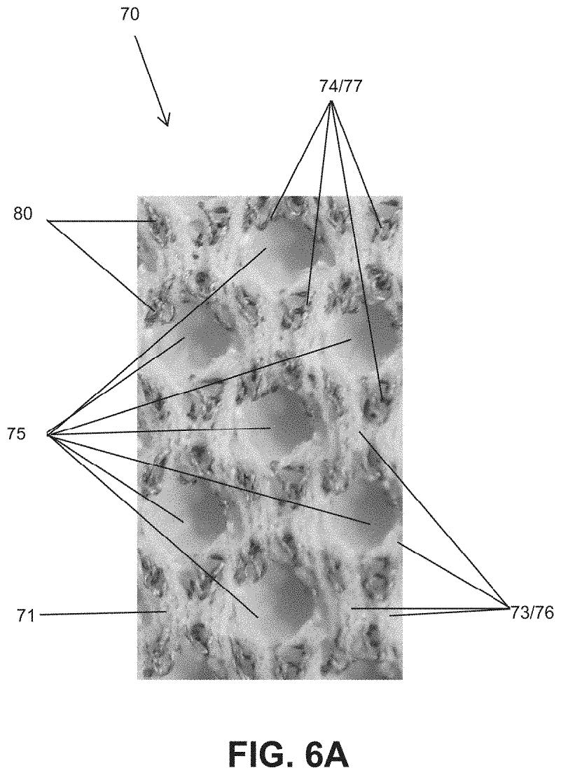

The support garment 50 may be configured for one-sided assembly of the electrode assembly 25 onto the support garment 50 such that the support garment 50 does not need to be flipped or turned over in order to properly position the therapy electrodes 11 and the sensing electrodes 12 on the support garment 50. The inside surface of the back portion 51 of the support garment 50 includes one or more pocket(s) 56a, 56b (together 56) for receiving one or two therapy electrodes 11 to hold the electrode(s) 11 in position against the patient's back. The one or more pockets 56 includes a mesh interface 70 (or mesh interfaces 70a, 70b) incorporating a plurality of dielectric fibers 73 comprising at least one nonmetallic material and a plurality of conductive fibers or particles 74 therein, as well as a plurality of openings 75 defined therein, as shown in FIGS. 6-8.

The mesh interface 70 is designed to physically separate the metallic therapy electrode(s) 11 from the skin of the patient P while allowing a conductive gel that may be automatically extruded from a plurality of holes 61 in the electrode(s) 11 to easily pass through to the skin of the patient P. The forces applied to the electrode(s) 11 by the mesh interface 70, in addition to the use of the conductive gel, may help ensure that proper contact and electrical conductivity with the patient's body are maintained, even during body motions. The mesh interface 70 also maintains electrical contact between the electrode(s) 11 through the material of the mesh interface 70 before the conductive gel is dispensed, which allows for monitoring of the therapy electrode(s) 11 to ensure that the electrode(s) 11 are positioned against the skin such that a warning may be provided by the wearable defibrillator 14 if the therapy electrode(s) 11 is not properly positioned. Another pocket, front pocket 57 including a mesh interface 70c according to the same construction is included on an inside surface of the belt 52 for receiving a front therapy electrode 11c and holding the electrode 11 in position against the patient's left side.

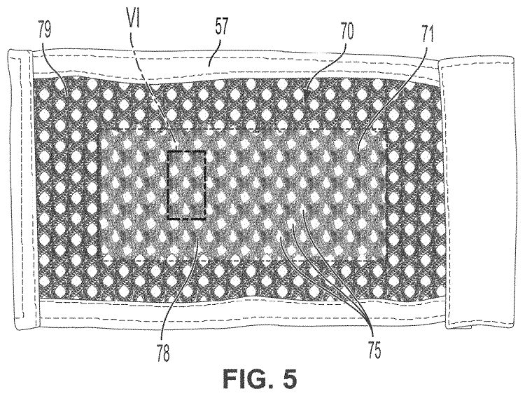

After assembly of the therapy electrode(s) into the respective pocket(s) 56, 57, the pocket(s) 56, 57 are closed on the support garment 50, by a fastener or fasteners 60, such as a button or snap. Further details regarding the mesh interfaces 70a, 70b, 70c of the pockets 56, 57 will be discussed in detail below with reference to FIGS. 5-10. In FIG. 4A, two rear pockets 56a and 56b, and one front pocket 57 are shown corresponding to the two rear therapy electrodes 11a and 11b, and front therapy electrode 11c. In other implementations, fewer or more rear or front pockets and/or therapy electrodes may be provided. For example, a garment can include two rear pockets and two front pockets, these pockets configured to receive two rear therapy electrodes and two front therapy electrodes. For example, a garment can include three rear pockets and three front pockets, these pockets configured to receive three rear therapy electrodes and three front therapy electrodes. In such examples, the rear or front pockets can include corresponding mesh interface as described herein.

The back portion 51 and the belt 52 of the support garment 50 may further incorporate attachment points 58 for supporting the sensing electrodes 12 in positions against the patient's skin in spaced locations around the circumference of the patient's chest. The attachment points 58 may include hook-and-loop fasteners for attaching ECG sensing electrodes 12 having a corresponding fastener disposed thereon to the inside surface of the belt 52. The attachment points 58 may be color coded to provide guidance for appropriately connecting the sensing electrodes 12 to the support garment 50. Additionally or alternatively, one or more of the ECG sensing electrodes can be permanently integrated into the belt 52 of the support garment 50, e.g., such that they cannot be removed/replaced by a patient during use. The support garment 50 may further be provided with a flap 59 extending from the back portion 51. The flap 59 and the back portion 51 include fasteners 60 for connecting the flap 59 to the inside surface of the back portion 51 in order to define a pouch or pocket for holding an processing and/or vibrational circuitry unit 13 of the electrode assembly 25. For example, the processing and/or vibrational circuitry unit 13 can include ECG acquisition and conditioning circuitry configured to receive ECG signals from the plurality of ECG sensing electrodes 12, amplify the signals, condition (e.g., using filter circuits) to remove noise, and sample the signal to produce a digitized ECG signal corresponding to the analog ECG input. In examples, the unit 13 can also include vibrational circuitry configured to receive an input from a controller (e.g., controller 120 shown in FIG. 9 below) and provide the patient a vibrational alarm or notification as appropriate. The outer surface of the belt 52 may incorporate a schematic 30 (shown in FIG. 2) imprinted on the fabric for assisting the patient or medical professional in assembling the electrode assembly 25 onto the support garment 50.

With reference to FIGS. 4A-10, according to an example of the present disclosure, the support garment 50 may be incorporated into a wearable cardiac therapeutic device for improved skin comfort when worn by a patient.

The device includes at least one therapy electrode 11 (for example, as shown here, two rear therapy electrodes and one front therapy electrode) configured to deliver therapeutic electrical pulses to a patient's heart; and the support garment 50 configured to support and hold the plurality of therapy electrodes 11 against the patient's body. The support garment 50 includes at least one support pocket 56, 57 (for example, as shown here, two rear pockets and one front pocket) disposed on an inside surface of the support garment 50 for supporting the at least one therapy electrode 11 on the support garment. A mesh interface 70 is formed as part of the each support pocket 56, 57. The mesh interface 70 is configured to facilitate electrical contact between the at least one therapy electrode 11 and the patient's skin.

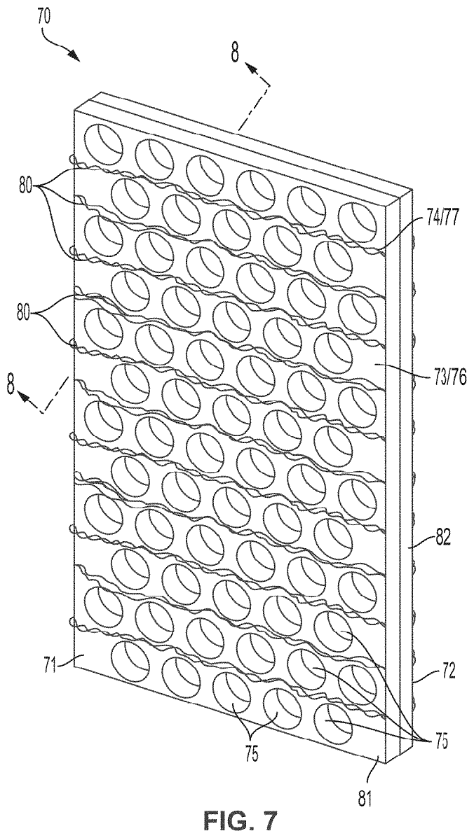

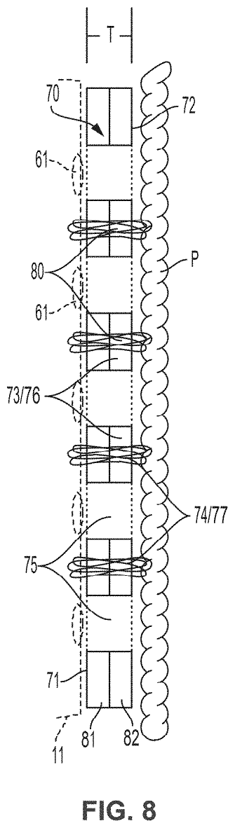

Referring briefly to FIGS. 5-8, the mesh interface 70 includes a first surface 71 oriented toward the at least one therapy electrode 11 and a second surface 72 oriented toward the patient's skin. The mesh interface 70 includes a plurality of dielectric fibers 73 comprising at least one nonmetallic material, and a plurality of conductive fibers and/or conductive particles 74. The plurality of conductive fibers and/or conductive particles 74 is interspersed and/or intertwined within and/or between the pluralities of dielectric fibers 73 to form a plurality of conductive pathways 80 extending through the mesh interface 70 from the first surface 71 to the second surface 72. The plurality of conductive pathways 80 are configured to conduct the therapeutic electrical pulses through the mesh interface 70 from the at least one therapy electrode 11 to the patient P. In some examples, the conductive pathways 80 project from the first surface 71 and the second surface 72 of the mesh interface 70, as shown in FIGS. 7 and 8, so as to be able to establish electrical contact with the therapy electrode 11 and the patient's skin P, respectively. In examples, the mesh interface 70 may also be configured to transmit a one or more electrical signals (e.g., therapy electrode “fall off” signal) from the at least one therapeutic electrode 11 to the patient's skin. For example, the therapy electrode “fall off” signal can be used by the controller 120 to determine that the at least one therapeutic electrode is present and/or correctly positioned on the patient's body.

According to an example, the plurality of conductive fibers and/or conductive particles 74 are interspersed within the mesh interface 70 in a concentrated manner at distinct locations throughout at least a portion of the mesh interface 70 to form sufficient conductive pathways 80 extending through the mesh interface 70 from the first surface 71 to the second surface 72. As described below, the mesh 70 is configured to comprise conductive pathways 80 distributed such that the mesh interface 70 has a sufficiently low impedance to allow for the therapeutic electrical pulses to be conducted from the at least one therapeutic electrode 11 to the patient's skin P. According to the example, the amount of conductive metallic material incorporated into the mesh interface 70 is reduced in comparison to a mesh fabric coated entirely with conductive metallic material. According to the example, the reduced amount of conductive metallic material incorporated in the mesh interface 70 allows for the inclusion of the dielectric, nonmetallic fibers 73 into the mesh interface 70.

According to an example, the conductive fibers and/or conductive particles 74 comprise a conductive yarn 77, such as a silver-plated nylon yarn, which is interlaced, such as by knitting, with dielectric fibers 73 comprised of a dielectric yarn or yarns 76, as will be discussed in further detail below with reference to FIGS. 5-8 and 10. According to another example, the conductive fibers and/or conductive particles 74 may be comprised of a liquid or gel based coating, i.e., paint, powder coating, and/or other fine material coating of conductive material applied to a layer or layers of dielectric material in a concentrated manner at select locations to saturate the dielectric material through its thickness. According to another example, the conductive fibers and/or conductive particles 74 comprise concentrated tufts or wads of a conductive yarn or other conductive fabric material inserted or plugged into a dielectric fabric material so as to extend through the thickness of the dielectric fabric material. According to another example, the conductive fibers and/or conductive particles 74 comprise conductive wires inserted into, such as by embroidering, a textile or fabric substrate.

According to an example of the present disclosure, the mesh interface 70 is configured to provide an electrical impedance of the plurality of conductive pathways 80 extending through the mesh interface 70 from the first surface 71 to the second surface 72 of approximately 0.01Ω-20Ω. In some examples, the range is approximately 0.01Ω-10Ω, or more particularly approximately 0.01Ω-5Ω, or more particularly approximately 0.1Ω-2Ω, or more particularly approximately 0.25Ω-1.5Ω. It is to be appreciated that the mesh interface 70 may provide any suitable electric impedance of the plurality of conductive pathways 80.

The electrical impedance values noted herein can be determined based on a test carried out in a following manner (“mannequin test”). For example, as shown in FIGS. 9A and 9B, a garment 1600 implementing the mesh interface 1603 can be positioned over a mannequin 1602. The mesh interface material 1603 shall be new and unwashed. To perform the test, the garment 1600 is placed on the mannequin 1602 as shown in FIGS. 9A and 9B. Therapy electrodes 1604 are placed in front 57 and rear pockets 56a that each include mesh interfaces 1603. A conductive pad 1606 is then placed under the garment 1600, between the garment 1600 and the surface of the mannequin 1602, such that the conductive pad 1606 is disposed between the mesh interfaces 1603 and the surface of the mannequin 1602. The conductive pad 1606 is connected by an electrical connector 1609 to wires to complete a circuit 1607 with one of the therapy electrodes 1604, which is also connected by an electrical connector 1609 to the wires forming the circuit 1607. Initially, a test current can be applied to the circuit. For example, the test current can be of predetermined suitable current parameters determined by circuit design. For example, the test current can comprise 0.01 A within a range of 200 Hz-200 MHz. Prior to an impedance measurement, conductive gel in a predetermined suitable quantity is deployed between the therapy electrode and the conductive pad as noted below. An ohmmeter and/or an A.C. bridge 1608 can be connected within the circuit 1607 and configured to measure the resistance of the mesh interface 70. In one scenario, the test can be performed at room temperature which corresponds to normal operating circumstances of the device. In some scenarios, the test can be repeated at a range of temperatures corresponding to usual ambient and/or typical patient use environments. For example, the test can be performed at temperatures ranging from −15 F to around 120 F. For example, the test can be performed at between 20% and 95% relative humidity. In some scenarios, typical ranges for carrying out the test can be within the range of 0° C. to 50° C. (32° F. to 122° F.), up to 95% relative humidity (non-condensing), and between sea level and up to 10,000 feet in altitude. The above-mentioned mannequin test is implemented to test the impedance each of mesh interface 1603 individually. It is to be appreciated that similar tests may be performed to test the impedance values of two or more mesh interfaces 1603 collectively.

According to an example, the plurality of conductive fibers and/or conductive particles 74 comprises an impedance measure of approximately 10-250 W/meter, or more particularly approximately 20-150 W/meter, and or more particularly approximately 30-130 Ω/meter. It is to be appreciated that the plurality of conductive fibers or particles 74 may comprise an impedance measure of any suitable value.

The mesh interface 70 is configured to provide a comfortable feel on the patient's skin and to wick moisture away from the patient's skin. As discussed above, the implementations herein include conductive metallic materials incorporated into the mesh interface 70 to reduce abrasion and irritation of the patient's skin, particularly during continuous use of the support garment 50 during an extended period of time. Also, such implementations may cause fewer negative reactions, such as an allergic reactions, to the metallic conductive materials. According to the example, the mesh interface 70 incorporates a reduced amount of the conductive metallic material, thereby reducing the potential for abrasion and irritation of the patient's skin, as well as the potential for negative reactions in response to prolonged contact with the mesh interface 70. In examples, the metallic material comprises silver metal. For example, silver metal comprises better conductivity than most other conductive metals, and further is more tolerable on skin than other conductive metals. In examples, silver comprises natural antimicrobial properties. Examples of such conductive materials for use in mesh interface as described in further detail below.

According to an example, the nonmetallic material of the plurality of dielectric fibers 73 comprises one or a combination of nylon, polyester, or cotton fibers. The dielectric fibers incorporated into the mesh interface 70 are configured preferably to have a soft, comfortable feel and are able preferably to absorb moisture on the patient's skin and wick the moisture away from the patient's skin, which further reduces the potential for abrasion and irritation of the patient's skin. Additionally, the nylon, polyester, and/or cotton fibers are more breathable than the conductive fibers or particles 74 and trap less heat against the patient's skin, which increases overall comfort to the patient P. The nylon, polyester, and/or cotton fibers may also protect the conductive fibers or particles 74 from wear and external damage, which may prolong the operational life of the support garment 50. According to an example, the dielectric fibers 73 comprise a textured nylon yarn, which creates a soft surface at the first surface 71 and the second surface 72 of the mesh interface 70.

According to an example, the nonmetallic material of the plurality of dielectric fibers 73 comprise fusible fibers, such as a fusible yarn, in combination with the above-mentioned nylon, polyester, or cotton fibers. The fusible fibers are configured to melt, dissipate, and/or shrink in volume relative to the conductive fibers or particles 74 and the other dielectric fibers 73 when exposed to heat, such as heat from steam generated by a garment steamer at approximately 70° C.-160° C. According to an example, the fusible fibers comprise a low melt, thermoplastic material, such as low melt nylon and/or low melt polyester materials. According to an example, the fusible fibers comprise a fusible bonding yarn formed from low melt nylon and/or low melt polyester multifilaments.

The fusible fibers provide for a good hand feel to the mesh interface 70. Also, as will be discussed in additional detail below, heating of the fusible fibers, thus causing the fusible fibers to melt, dissipate, and/or shrink in volume, results in the conductive fibers or particles 74 expressing more relative to the dielectric fibers 73, whereby the plurality of conductive pathways 80 extending through the mesh interface 70 project from the first surface 71 and the second surface 72 of the mesh interface 70, as shown in FIGS. 7 and 8. According to the example, the shrinkage of the dielectric fibers 73 and the projection of the conductive pathways 80 from the first surface 71 and the second surface 72 of the mesh interface 70 allows for the number of conductive fibers or particles 74 to be reduced within the mesh interface 70 while achieving the sufficiently low impedance to effectively transmit the therapeutic electrical pulses from the therapy electrodes 11 to the patient P. The reduction in the amount of conductive fibers or particles 74 in the mesh interface allows for increased comfort and the potential for less skin irritation experienced by the patient P.

According to one example of the present disclosure, the mesh interface 70 is comfortable so as to not cause human skin irritation after predetermined test periods as set forth below (e.g., after 1 day of continuous contact exposure, after 2 days of continuous contact exposure, or after 3 days of continuous contact exposure). For instance, in some examples, the mesh interface 70 is constructed so as to score zero or no more than one on the Human Skin Irritation Test set forth in Annex C of the ANSI/AAMI/ISO 10993-10:2010 standards for Biological Evaluation of Medical Devices—Part 10: Tests for Irritation and Skin Sensitization, the contents of which are hereby incorporated by reference. Table C.1 of Annex C, which provides the grading scale for the Human Skin Irritation Test, is set forth below. In accordance with ISO 10993-10 C3.3., at least 30 volunteers shall complete the test, with no less than one-third of either sex. The mesh interface test material shall be applied to intact skin at a suitable site, e.g. the upper outer arm. The application site shall be the same in all volunteers and shall be recorded. Generally, the mesh interface test material shall measure at least 1.8 cm, preferably 2.5 cm in diameter. The mesh interface test material shall be held in contact with the skin by means of a suitable non-irritating dressing, including non-irritating tape, for the duration of the exposure period. In one scenario, the mesh interface test material can be pre-moistened with water before application. To avoid unacceptably strong reactions, a cautious approach to testing shall be adopted. A sequential procedure permits the development of a positive, but not severe, irritant response. The mesh interface test materials are applied progressively starting with durations of 15 min and 30 min, and up to 1 h, 2 h, 3 h and 4 h. The 15 min and/or 30 min exposure periods may be omitted if there are sufficient indications that excessive reactions will not occur following the 1 h exposure. If no reaction or no excessive reactions are observed, the duration can be increased to 1 day, 2 days, and 3 days. Progression to longer exposures, including 24 h exposure at a new skin site, will depend upon the absence of skin irritation (evaluated up to at least 48 h) arising from the shorter exposures, in order to ensure that any delayed irritant reaction is adequately assessed.

Application of the material for a longer exposure period is always made to a previously untreated site. At the end of the exposure period, residual test material shall be removed, where practicable, using water or an appropriate solvent, without altering the existing response or the integrity of the epidermis. Treatment sites are examined for signs of irritation and the responses are scored immediately after mesh interface test material removal and at (1±0.1) h to (2±1) h, (24±2) h, (48±2) h and (72±2) h after patch removal. If necessary to determine reversibility of the response, the observation period may be extended beyond 72 h. In addition, the condition of the skin before and after the test shall be thoroughly described (e.g. pigmentation and extent of hydration). Skin irritation is graded and recorded according to the grading given in Table C.1 of Annex C.

TABLE C.1
Human skin irritation test, grading scale

| Description of response | Grading |
|---|---|
| No reaction | 0 |
| Weskly positive reaction (usually characterized by mild | 1 |
| erythema and/or dryness across most of the treatment site) | |
| Moderately positive reaction (usually distinct erythema or | 2 |
| dryness, possibly spreading beyond the treatment site) | |
| Strongly positive reaction (strong and often spreading | |
| erythema with oedema and/or eschar formation) | 3 |

The mesh interface 70 may also be configured to facilitate a transfer of conductive gel from the at least one therapy electrode to the patient's skin. As shown in FIGS. 5-8, the mesh interface 70 may further comprise a plurality of openings 75 extending through the mesh interface 70 from the first surface 71 to the second surface 70. The mesh interface 70 is configured to facilitate transfer of conductive gel from the at least one therapy electrode 11 to the patient's skin via the plurality of openings 75.

According to an example, the mesh interface 70 is configured to receive conductive gel from the plurality of holes 61 in the at least one therapy electrode 11 in an amount of approximately 0.1 cubic-centimeter (cc) to 100 cc of conductive gel, or approximately 0.1 cubic-centimeter (cc) to 75 cc of conductive gel, or approximately 0.1 cubic-centimeter (cc) to 30 cc of conductive gel. In examples, the mesh interface 70 is configured to receive conductive gel from a plurality of holes 61 in an amount of approximately 0.5 cc to 20 cc, or more particularly approximately 0.9 cc to 10 cc, and 0.9 cc to 5 cc. It is to be appreciated that the mesh interface 70 may be configured to receive any suitable amount of the conductive gel. FIG. 8 depicts the plurality of holes 61 of the therapy electrode 11 in alignment with the openings 75 of the mesh interface. This is depiction is provided as an example and done for ease of illustration and to demonstrate the purpose of the openings 75 to facilitate transfer of conductive gel from the openings 61 in the therapy electrode 11 to the patient P. It is not necessary for the mesh interface 70 to be configured in such a manner such that the plurality of openings 75 in the mesh interface align with the plurality of openings 61 in the therapy electrode.

According to an example, the conductive gel is configured to provide a predetermined electrical impendence of the plurality of conductive pathways extending through the mesh interface 70 from the first surface 71 to the second surface 72 of approximately 0.01Ω-10Ω. In some examples, the range is approximately 0.01Ω-5Ω, or more particularly approximately 0.1Ω-2Ω, and or more particularly approximately 0.25Ω-1.5Ω. It is to be appreciated that the conductive gel may be configured to provide a predetermined electrical impedance of the plurality of conductive pathways of suitable value.

For example, the conductive gel can have a viscosity of around 2000 centipoise (cps) to 70,000 cps. In some examples, the range is approximately 10,000-50,000 cps, or more particularly approximately between 20,000-45,000 cps. The viscosity can be determined based on a standard Viscometer, such as a Brookfield Synchro-Lectric Viscometer, at around a temperature range of between 65-100 F. For example, the resistance of the conductive gel can be in a range of 0.01Ω-20Ω, or more particularly in a range of approximately 3Ω-15Ω, as measured using a standard A.C. bridge circuit, such as a Belco A.C. Bridge with electrodes located at around 1-2 inches apart and immersed within the conductive gel, at around a temperature range of between 65-100 F.

According to an example, the mesh interface 70 is configured to be porous to the conductive gel from a plurality of holes 61 in the at least one therapy electrode 11 to provide a predetermined electrical impendence of the plurality of conductive pathways 80 extending through the mesh interface 70 from the first surface 71 to the second surface 72 of approximately 0.01Ω-20Ω. In some examples, the range is approximately 0.010-10Ω, or more particularly approximately 0.01Ω-5Ω, or more particularly approximately 0.1Ω-2Ω, and or more particularly approximately 0.25Ω-1.5Ω. It is to be appreciated that the mesh interface 70 may be porous to the conductive gel to provide a predetermined electrical impedance of any suitable value. The electrical impedance values noted herein can be determined based on a test carried out in a manner that is similar to that described above as the mannequin test.

The plurality of openings 75 are provided in the mesh interface 70 in a sufficient number and have a sufficient size to facilitate the transfer of a suitable amount of the conductive gel from the at least one therapy electrode 11 to the patient's skin to achieve an appropriate level of impedance (or alternatively measured as admittance, which is the inverse of impedance) between the at least one therapy electrode 11 (via the mesh interface 70) and the patient's skin such that the therapeutic electrical pulses are delivered to the patient's heart without burning or with minimal burning of the patient's skin. For example, the appropriate level of impedance between the at least one therapy electrode 11 via the mesh interface 70 and the patient's skin is tested per the mannequin test as noted above, and is approximately 0.01Ω-20Ω, including ranges therebetween as noted above.

According to an example, the plurality of openings comprises approximately 2-1000 openings 75 per square inch of the mesh interface 70, or more particularly approximately 5-500 openings 75 per square inch of the mesh interface 70, and or more particularly approximately 10-100 openings 75 per square inch of the mesh interface 70. It is to be appreciated that the plurality of openings 75 may comprise any suitable number of openings 75. According to an example, the plurality of openings 75 have an average diameter in a range of approximately 0.005″-0.3″ (0.13 mm-7.6 mm), or more particularly approximately 0.01″-0.2″ (0.25 mm-5.1 mm), or more particularly approximately 0.05″-0.1″ (1.3 mm-2.5 mm). It is to be appreciated that the plurality of openings 75 may have any suitable average diameter or range of varying average diameters.

According to an example, the plurality of openings 75 may have a non-circular shape, such as quadrilateral, rectangular, square, triangular, pentagonal, hexagonal, octagonal, etc. According to the example, the plurality of openings may have an average area in a range of approximately 0.01 mm²-45.4 mm², or more particularly 0.05 mm²-20.4 mm², or more particularly 1.3 mm²-4.9 mm².

The thickness T, shown in FIG. 8, of the mesh interface 70 may also effect the amount of the conductive gel transferred from the at least one therapy electrode to the patient's skin. The thickness T is measured prior to application to the patient's body as the mesh interface material will be compressed between the therapy electrode and patient's skin. According to an example, a thickness T of the mesh interface 70 (in uncompressed form) from the first surface 71 to the second surface 72, shown in FIG. 8, is approximately 0.005″-0.5″ (0.13 mm-12.7 mm), or more particularly approximately 0.01″-0.25″ (0.25 mm-6.4 mm), and or more particularly approximately 0.03″-0.1″ (0.76 mm-2.5 mm). It is to be appreciated that the mesh interface 70 may be provided with any suitable thickness T in order to carry out its functionality as described herein.

With reference to FIGS. 6-8 and 10, according to an example of the present disclosure, the plurality of dielectric fibers 73 of the mesh interface 70 comprise one or more dielectric yarns 76 and the plurality of conductive fibers and/or conductive particles 74 of the mesh interface 70 comprise one or more conductive yarn(s) 77. The dielectric yarn(s) 76 and the conductive yarn(s) 77 are intertwined together to form the mesh interface 70. It is to be appreciated that the dielectric yarn(s) 76 and the conductive yarn(s) 77 may be intertwined in any suitable manner. For instance, the mesh interface 70 may be formed by a knitting, weaving, and/or interlacing process, or any other suitable process for creating a fabric material. The process of intertwining the dielectric yarn(s) 76 and the conductive yarn(s) 77 may be performed automatically by a suitable machine.

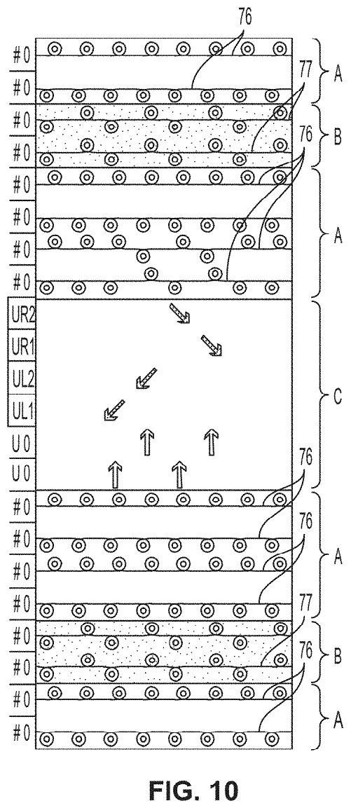

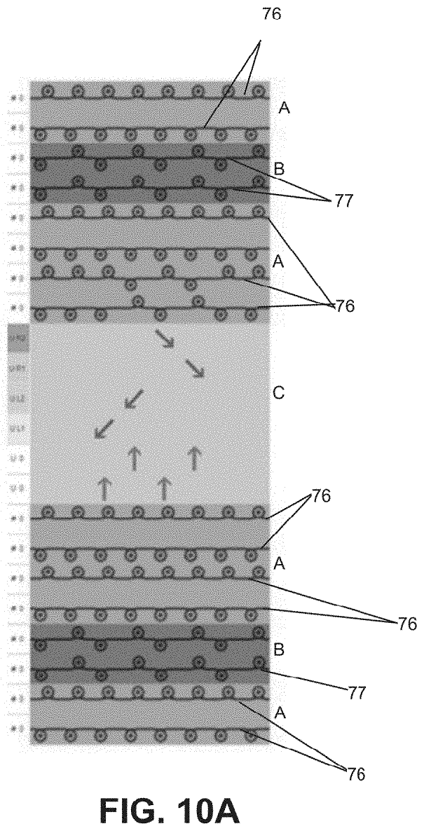

According to one example, the dielectric yarn or yarns and the conductive yarn can be flat knitted on E7.2 Stoll ADF32-W knitting machine having front and back needle beds in a V-Bed configuration, which is commercially available from STOLL of Reutlingen Germany. FIG. 10 is a schematic illustration of the knitting pattern structures/knitting process performed by the above-mentioned E7.2 Stoll ADF32-W machine to form the mesh interface 70 according to the present example. The schematic of FIG. 10 may be provided to the machine by an operator as a set of instructions for knitting the mesh interface 70 or generated by the machine according a set of coded instructions provided by the operator. It is to be appreciated that the mesh interface 70 may be machine knitted on any suitable machine according to any suitable technique, particularly on any other suitable 14 gage double bed flat knitting machine. FIG. 10 also illustrates an example racking (left side column of FIG. 10) of the needle beds machine knitting of the mesh interface 70 on the above-mentioned E7.2 Stoll ADF32-W machine.

In examples, the knitting machine is configured to be controlled by a processor executing a plurality of machine-readable instructions stored on a non-transitory computer-readable medium. In implementations, knitting yarn into a seamless knitted preform using a computerized flat knitting machine allows for variations in shapes that can be produced with a reduced amount of materials and parts waste, human effort, and time. For example, a user can design the therapy mesh interface in accordance with the principles described herein based on a three-dimensional shape using a computer-aided design (CAD) program. The design can then be knit into a seamless knitted preform by the computerized flat knitting machine such that multiple sheets of materials and yarn do not need to be manually cut and laid up to form the shape and structure of the therapy mesh interface. In examples, the processor can be disposed within a printed circuit board (PCB), and can comprise an integrated random operating memory (ROM) chip. As controlled by a custom run program stored on the processor to implement the therapy mesh interface described herein, the processor generates control signals to engage the drive motion in coordination with the mechanical spring arm of the knitting machine.

According to an example, the dielectric yarn(s) 76 comprises a textured nylon or cotton yarn and a fusible yarn. According to one example, the textured nylon or cotton yarn comprises 70/1/34 denier textured nylon yarn and the fusible yarn comprises 75 deci-tex/F75 fusible yarn. According to an example, the fusible yarn comprises a low melt, thermoplastic material, such as multifilaments of low melt nylon and/or low melt polyester, and is formed such that the fusible yarn begins to melt, dissipate, and/or shrink in volume when exposed to heat at 75° C.

According to an example, the conductive yarn 77 comprises a silver-plated nylon yarn. The silver-plated nylon yarn may be a 2 ply 100 denier conductive yarn with a maximum resistance of 75 W/meter. According to the example, the silver material is chosen for the conductive yarn 77 due to its conductivity and biocompatibility, i.e., reduced potential for skin irritation and negative reactions. According to the example, the silver-plated nylon yarn can be generated according to any plating or metal application technique for depositing the silver material on the nylon (or other polyamide) substrate material, including through the use of electrolysis, chemical reactants, bundle drawing, machining, lamination, foil-shaving, and/or metalizing/vapor deposition.

According to an example, a conductive yarn 77 different from the silver-plated nylon yarn may be intertwined with the dielectric yarn(s) 76. For instance, the conductive yarn 77 may comprise a nickel plated/metalized or aluminum plated/metalized nylon, other polyamide, or polytetrafluoroethylene (PTFE) yarn. The conductive yarn 77 may also comprise a carbon coated or carbon filled yarn or filament material. Alternatively, the conductive yarn 77 may comprise a yarn material that has been coated or painted with a conductive paint material, such as a silver loaded polymer paint.

According to an example, the mesh interface 70 comprises approximately 10%-60% by weight of conductive yarn 77, or more particularly approximately 15%-54% by weight of conductive yarn 77, and or more particularly approximately 20%-35% by weight of conductive yarn 77. It is to be appreciated that the conductive yarn 77 may be provided in any suitable amount or ratio. In particular, the mesh interface 70 incorporates a sufficient amount of conductive yarn 77 to form sufficient conductive pathways 80 extending through the mesh interface 70 from the first surface 71 to the second surface 72 such that the mesh interface 70 has a sufficiently low impedance to allow for the therapeutic electrical pulses to be conducted from the at least one therapeutic electrode 11 to the patient's skin P.

The impedance of the mesh interface 70 must be maintained over the operational life of the support garment 50 through continuous or nearly continuous use and multiple wash cycles. For reference, patients may be instructed to wash their support garments every day or nearly every day. Accordingly, 30 wash cycles represents an approximate typical number of wash cycles of the support garment for a month. Continuous use of the support garment and multiple wash cycles tend to result in the loss of conductive metal material from the mesh interface 70 over time, resulting in an increase in the impedance of the mesh interface 70 over time.

Examples of the mesh interface 70 having a higher weight percentage of the conductive yarn 77 may be associated with support garments 50 that are intended to have a longer operational life since the presence of a sufficient amount of conductive metal material can be maintained in the mesh interface 70 through continuous wear of the support garment 50 and a large number of wash cycles, i.e, 30 or more days/wash cycles.

Examples of the mesh interface 70 having a lower weight percentage of the conductive yarn 77 may be associated with support garments 50 that are intended to have a more limited operational life, i.e., 10-15 days/wash cycles, as the impedance of the mesh interface 70 will not be maintained over time due to the loss of the conductive material from the mesh interface after a short amount of time. According to an example, a support garment 50, which is intended to be worn for a limited amount of time, i.e., no more than 10-15 days, may be provided with a mesh interface 70 including a reduced weight percentage of the conductive yarn 77 to reduce cost and avoid waste.

As shown in FIG. 10, the mesh interface 70 may comprise a plurality of intertwined structures A, B, C of the dielectric yarn 76 and the conductive yarn 77. According to an example, the plurality of intertwined structures A, B, C comprises a pattern of at least three intertwined structures A, B, C. The at least three intertwined structures A, B, C comprise a plurality of courses of the dielectric yarn 76 arranged in a tubular pattern structure A; at least one course of the conductive yarn 77 intertwined with the plurality of courses of the dielectric yarn 76 in a 1×1 rib pattern structure B; and at least one pointelle pattern structure C of intertwined dielectric yarn. A “pointelle” is a knit fabric structure in which the knitting process forms a pattern of small holes in the finished fabric material.

As discussed above, the dielectric yarn 76 comprises the textured nylon or cotton yarn and the fusible yarn. The textured nylon or cotton yarn and the fusible yarn may be arranged together in a plated yarn structure. In other words, the textured nylon or cotton yarn and the fusible yarn may be fed to the needle bed together or in a slightly offset manner such that both yarns are knitted together course by course with one yarn, i.e., the textured nylon or cotton yarn, showing on the needle front (technical face) and one yarn, i.e., the fusible yarn, showing on the needle back (technical back).

As shown in FIG. 10, according to the example, the tubular pattern structure A includes six courses of the plated textured nylon or cotton yarn and fusible yarn 76 knitted in an alternating manner on the front and back needle beds, i.e., front-back-front-back-front-back. It is to be appreciated that this process results in 2 layers 81, 82 (shown schematically in FIGS. 7 and 8) of the plated textured nylon or cotton yarn and fusible yarn being knitted side by side (front and back) with the textured nylon or cotton yarns of the two layers 81, 82 forming the front and back of an exterior of the plurality of courses of dielectric yarn 76 in the tubular pattern structure A and the fusible yarns forming the interior of the plurality of dielectric yarn courses 76 in the tubular pattern structure A.

The at least one course of the conductive yarn 77 may include two courses of conductive yarn 77 arranged in a 1×1 rib pattern structure B that are intertwined with the plurality of courses of dielectric yarn 76 in the tubular pattern structure A such that the courses of conductive yarn 77 extend from the first surface 71 of the mesh interface 70 to the second surface 72 of the mesh interface 70. The 1×1 rib pattern structure B is configured such that the courses of conductive yarn 77 stand out of the first and second surfaces 71, 72 of the mesh interface 70. In particular, feeding of the dielectric yarn courses 76 may lead feeding of the conductive yarn courses 77. Therefore, the conductive yarn courses 77 will show on top of the dielectric yarn 76 on both sides of the mesh interface 70 and thereby stand out. The 1×1 rib pattern structure B connects the first and second surfaces 71, 72 every other stitch, thereby forming the plurality of conductive pathways 80 through the mesh interface 70, providing good electrical contact between the at least one therapy electrode 11 and the patient's skin P, and ensuring a sufficiently low impedance between the first and second surfaces 71, 72 of the mesh interface 70. The conductive yarn courses 77 in the 1×1 rib pattern structure B may be loosely knit so as to not stretch the conductive yarn and to allow the conductive yarn courses 77 to project from the first and second surfaces 71, 72 of the mesh interface 70.

The at least one pointelle pattern structure C may be a 4 needle pointelle and defines the plurality of openings 75 extending through the mesh interface 70 from the first surface 71 to the second surface 72. As shown in FIG. 10, the pointelle pattern structure C is formed between successive tubular pattern structures A by transferring stitches between the front needle bed and the back needle bed and racking certain needles in the back needle bed (i.e., moving the needles sideways) to move the stitches to the left or right to form an opening 75 in the mesh interface. In FIG. 10, the vertical arrows represent the transfer of a stitch between opposite needles in the front and back needle beds and the diagonal arrows represent the transfer of a stitch between a needle in the front needle bed and a needle in the back needle bed that has been racked two positions in the back needle bed. It is to be appreciated that the pointelle pattern structure C for forming the plurality of openings 75 in the mesh interface may be performed in any suitable manner.

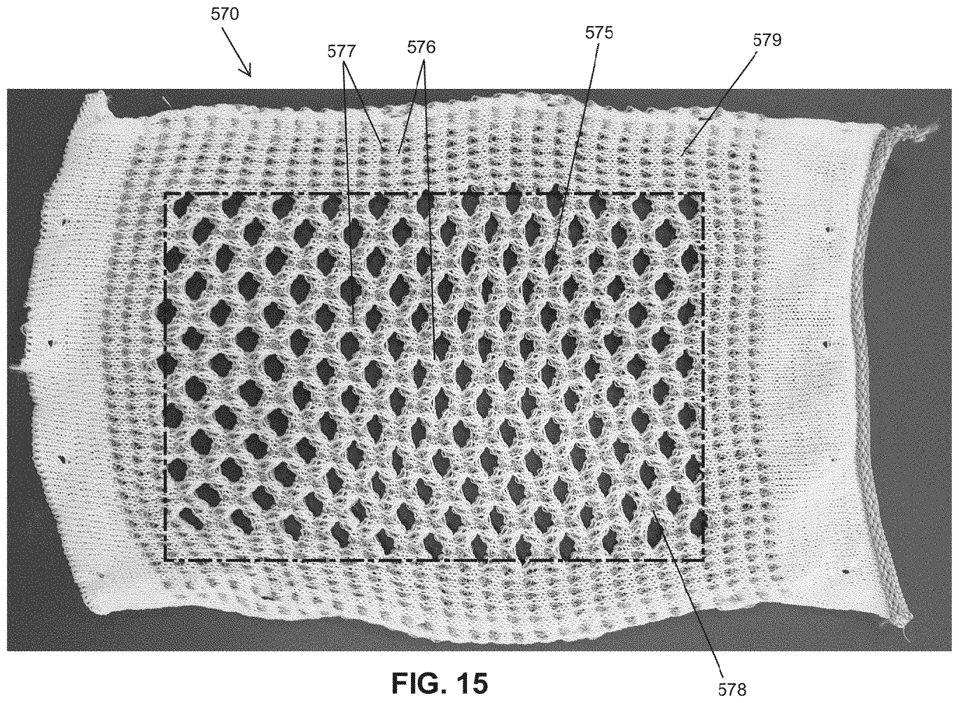

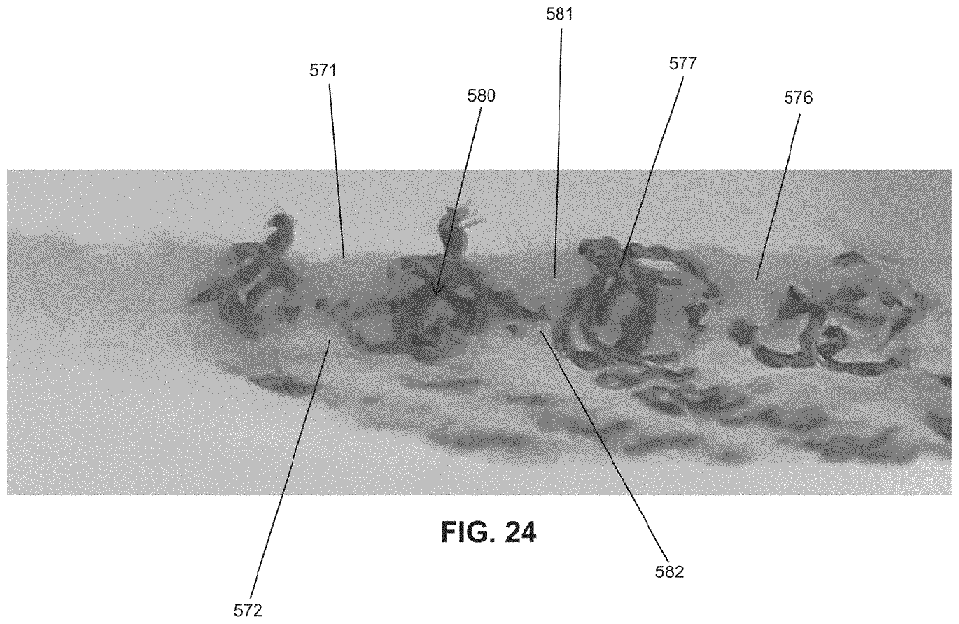

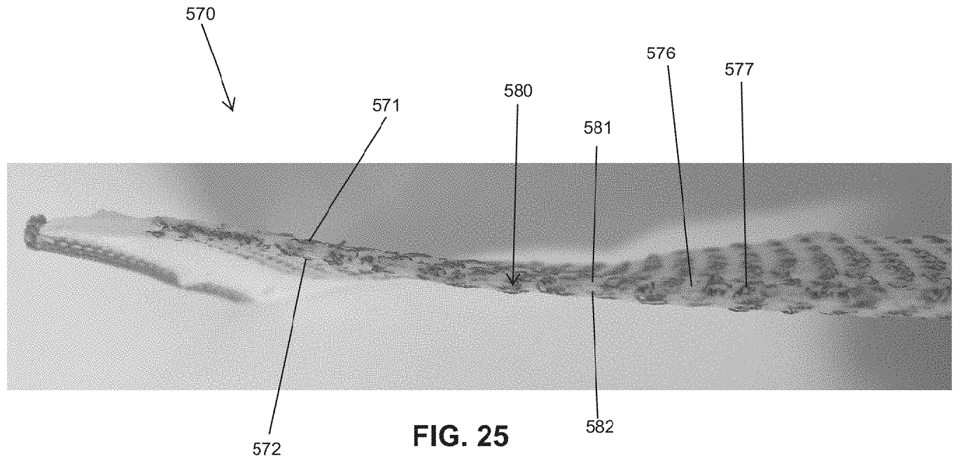

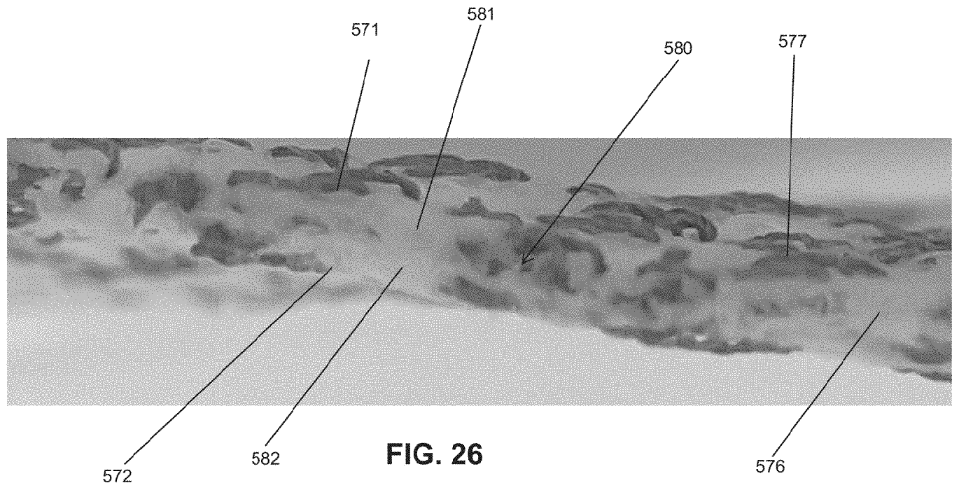

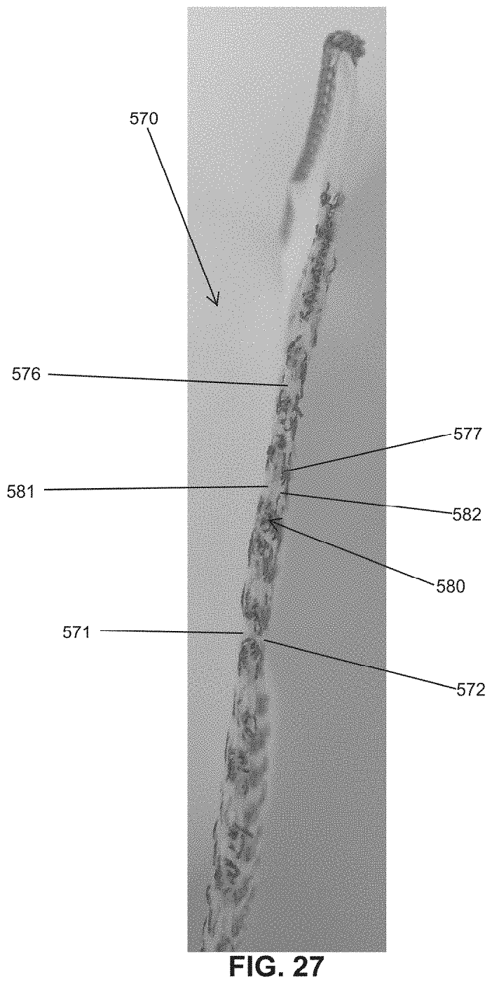

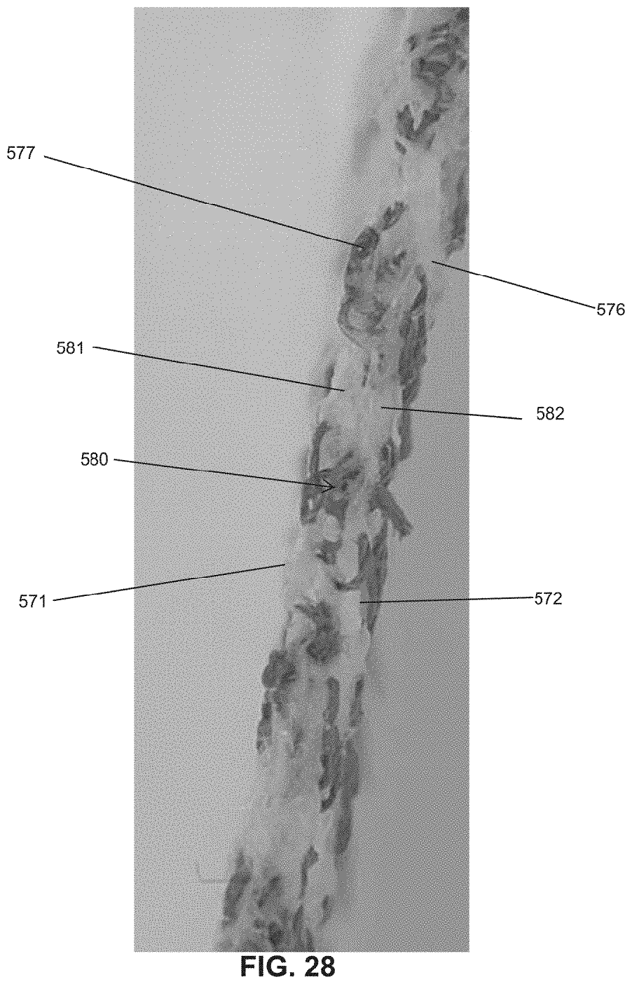

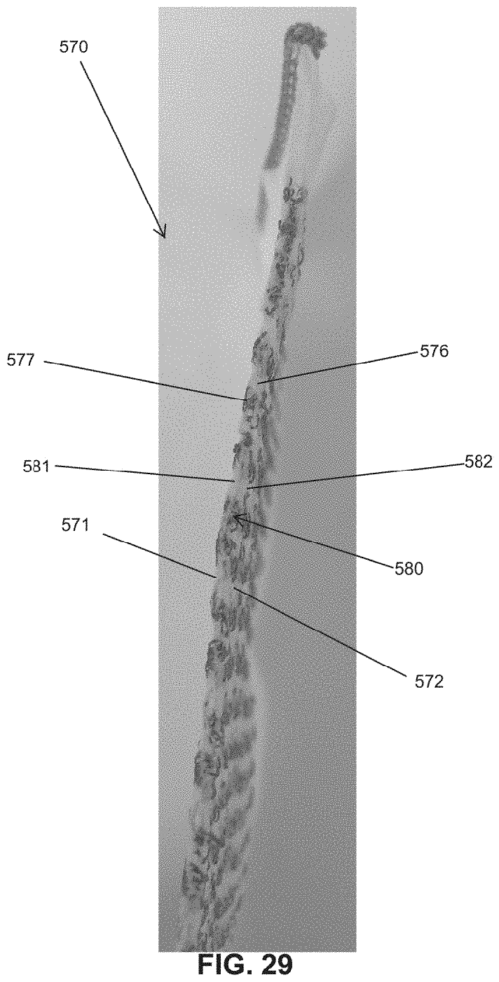

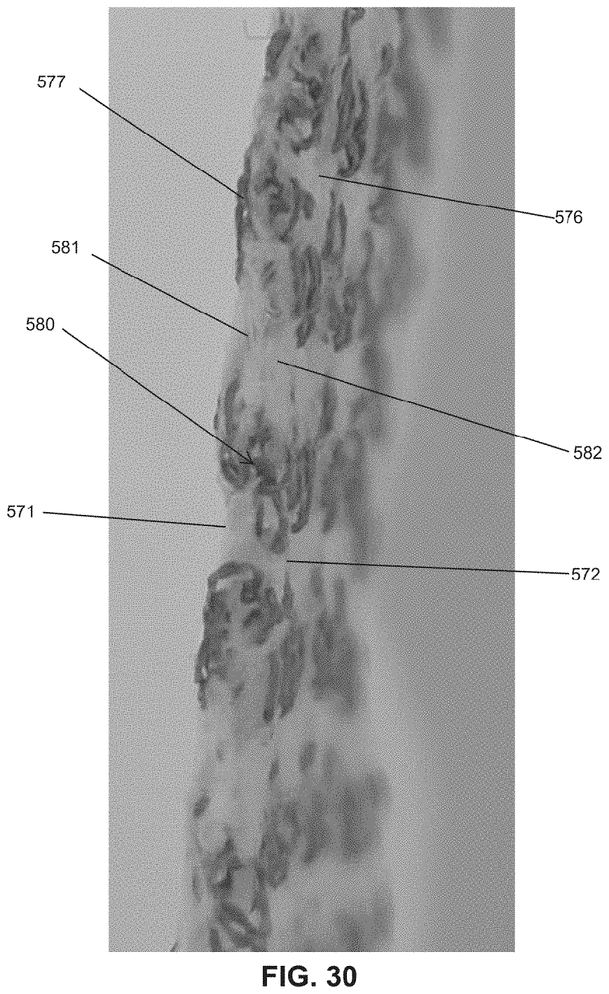

With reference to FIG. 5, according to an example, the mesh interface 70 may include a central portion 78 including the dielectric yarn(s) 76 and the conductive yarn 77 intertwined with each other in the tubular pattern structures A, the 1×1 rib pattern structures B, and the pointelle pattern structures C described above with reference to FIGS. 6 and 8-10. A peripheral portion 79 may be formed surrounding the central portion 78 that does not include the openings 75 formed by the pointelle pattern structures C. The peripheral portion 79 may include the dielectric yarn(s) 76 and the conductive yarn 77 intertwined with each other in the tubular pattern structures A and the 1×1 rib pattern structures B described above with reference to FIGS. 6 and 8-10, as will be described below with reference to FIGS. 15-31. The peripheral portion 79 of the mesh interface 70 may be less porous with respect to the passage of the conductive gel from the plurality of openings 61 in the therapy electrode 11 to the patient's skin P than the central portion 80 of the mesh interface 70, while remaining configured to conduct the therapeutic electrical pulses from the therapy electrode 11 to the patient P.

According to another example, the peripheral portion 79 may not include any conductive yarn 77 or conductive fibers and/or conductive particles 74 and may not contribute to conduction of the therapeutic electrical pulses from the therapy electrode 11 to the patient P. Rather, the peripheral portion 79 may be formed entirely from dielectric materials. According to the example, the peripheral portion 79 may be formed from the textured nylon or cotton yarn and fusible yarn intertwined as described above with reference to FIG. 10 and incorporating a pointelle pattern to form openings in the peripheral portion 79 that allow for conductive gel to pass through the peripheral portion 79 from the therapy electrode 11 to the patient's skin P. Alternatively, the peripheral portion 79 may be formed from an entirely different knitted pattern structure surrounding the central portion 78 or from an entirely different fabric material that the central portion 78 can incorporated into, such as by stitching, as a patch. According to another example, the entire mesh interface 70 is formed entirely of the tubular pattern structures A, the 1×1 rib pattern structures B, and the pointelle pattern structures C described above with reference to FIG. 10.

In implementations, each mesh interface 70 can be constructed in a different manner or each mesh interface 70 can be identical. For example, referring to FIG. 4A, one or both rear mesh interfaces 70a and 70b can be implemented with more metallic material than the front mesh interface 70c. For example, referring to FIG. 4A, one or both rear mesh interfaces 70a and 70b can be implemented metallic material and no dielectric material, while the front mesh interface 70c can be implemented with a mix of metallic material and dielectric material as described herein. According to an example, the mesh interfaces 70a, 70b of the support pockets 56a, 56b in the back portion 51 of the support garment 50 are made entirely from conductive yarn and the mesh interface 70c of the support pocket 57 in the belt portion 52 of the support garment is formed according to the structure described herein with reference to FIGS. 5-11B. Alternatively, the mesh interfaces 70a, 70b in the back portion 51 may include a higher content of conductive yarn than the mesh interface 70a in the belt portion 52 to achieve a lower impedance. Such implementations may be more feasible without increasing the skin irritation of the patient P, because the back portion 51 of the support garment 50 is less likely to move with respect to the patient's skin than the belt portion 52, which tends to be shifted in position as the patient lies down, sits, stands, raises his/her arms, moves, bends, etc., thus causing additional friction between the mesh interface 70 and the patient's skin.

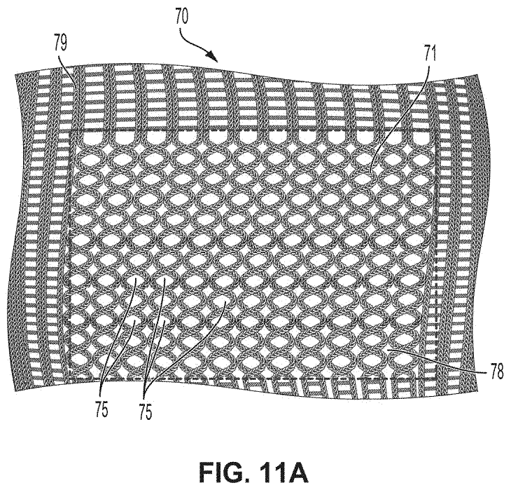

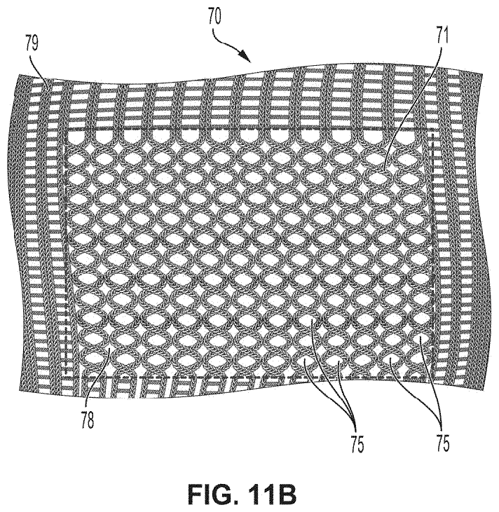

With reference to FIGS. 10, 11A and 11B, as discussed above, the dielectric fibers 73 of the mesh interface 70 include a textured nylon or cotton yarn and a fusible yarn that is configured to melt, dissipate, and/or shrink in volume relative to the nylon or cotton yarn and the conductive yarn when exposed to heat, such as steam generated by a garment steamer at 70° C.-160° C. Accordingly, heating of the dielectric yarns of the mesh interface causes the fusible yarn to melt, dissipate, and or shrink in volume thereby contracting the structures A of dielectric yarns 76 relative to the structures B of conductive yarn 77 in the mesh interface 70, which enhances the standing out of the conductive yarn 77 from the first and second surfaces 71, 72 of the mesh interface 70, as shown in FIGS. 7 and 8.

FIG. 11A illustrates an example stretched mesh interface 70 prior to being exposed to an application of heat during design and manufacture. The textured nylon or cotton yarns and the fusible yarns are relatively relaxed prior to the application of heat. FIG. 11B illustrates the example mesh interface 70 after being stretched and exposed to an application of heat. The fusible yarns have melted, dissipated, and or shrunk in volume, thus contracting the dielectric yarn structures A, which has the effect of widening the plurality of openings 75, fusing the loosely knit structures B of the conductive yarn, which is not subject to the same shrinkage effects under heat, closer together within the dielectric yarn structures A, and causing the structures B of the conductive yarn to further stand out from the first and second surfaces 71, 72 of the mesh interface 70 such that the conductive pathways 80 further project from the first and second surfaces 71, 72 of the mesh interface. This effect allows for greater electrical connectivity between the conductive fibers or particles 74 interspersed within the mesh interface 70, thereby resulting in an increased plurality of conductive pathways through the mesh interface 70 from the first surface 71 to the second surface 72. For example, the heat process can cause the conductive fibers or particles to express more relative to the dielectric fibers. The dielectric fibers recede during the process to be sub-flush relative to the conductive fibers. This process allows for fewer conductive fibers or particles to be used in the development of the mesh interface. Accordingly, stretching and application of heat to the mesh interface 70 may be utilized to reduce conductive fibers or particles, and improve transfer of conductive fluid and conduction of therapeutic electrical pulses through the mesh interface 70 from the at least one therapy electrode 11 to the patient P.

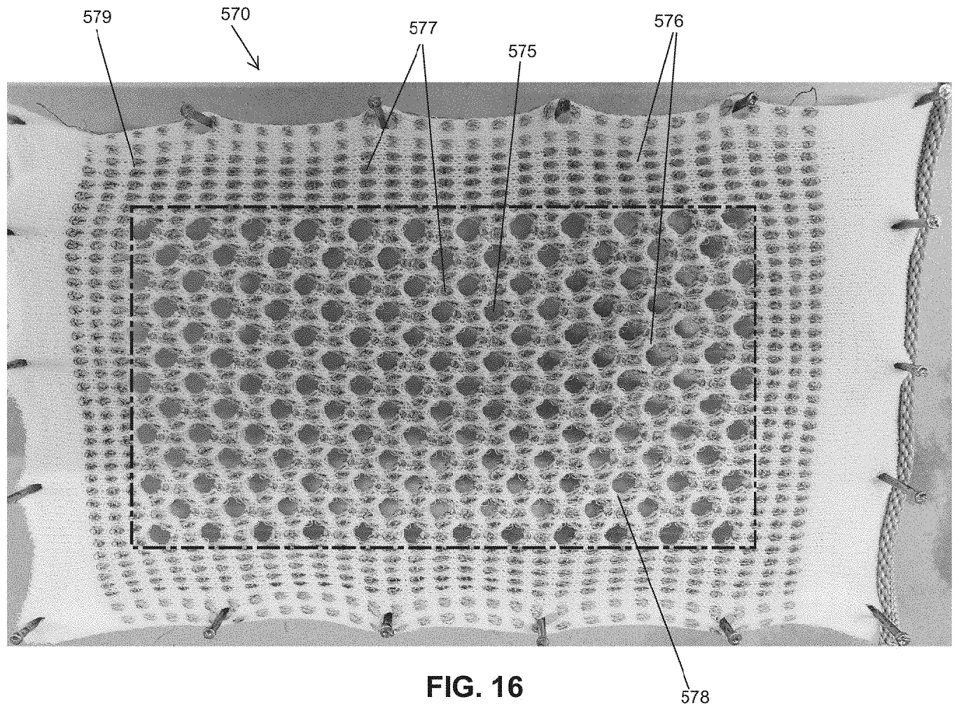

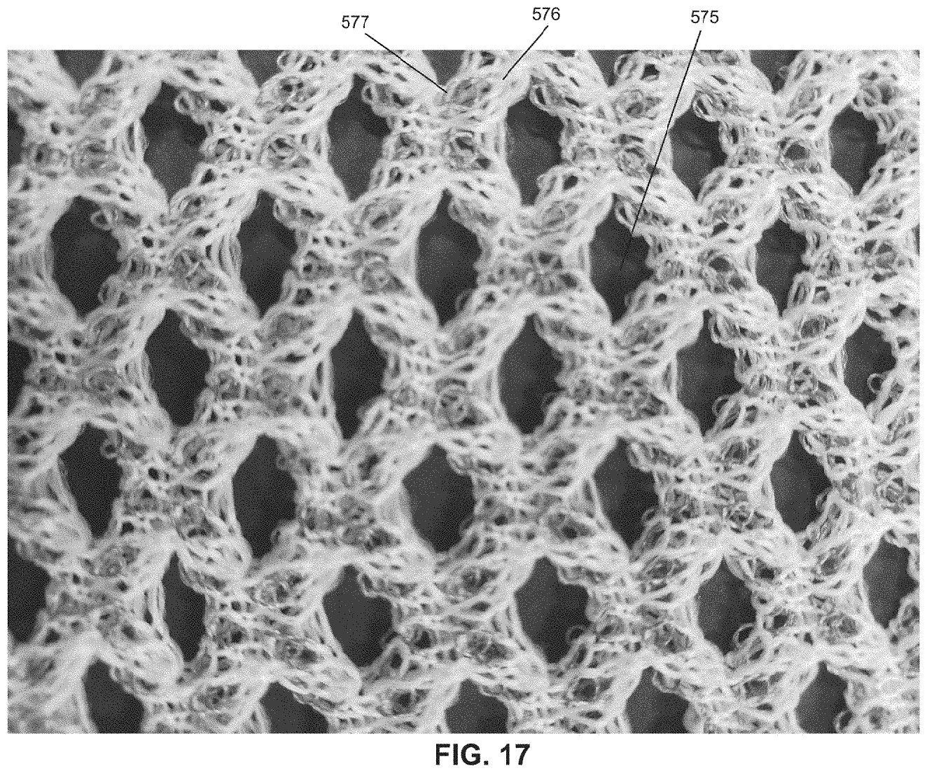

With reference to FIGS. 15-31, a sample mesh interface 570 is shown that has been formed according to the techniques described herein with reference to FIGS. 5-11B. FIGS. 15-18 illustrate the sample mesh interface 570 after the mesh interface 570 has been knitted by a E7.2 Stoll ADF32-W machine according to the pattern/instructions set forth in FIG. 10, as discussed above. FIG. 15 illustrates the sample mesh interface 570 in an unstretched condition. FIG. 17 illustrates an enlarged portion of the sample mesh interface 570 shown in FIG. 15. FIG. 16 illustrates the sample mesh interface 570 in a stretched condition. As shown, the sample mesh interface 570 includes a central portion 578 that includes the dielectric yarns 576 comprised of a textured nylon yarn plated with a fusible thermoplastic yarn arranged in the tubular pattern structures A described above, the conductive yarn 577 loosely intertwined with the dielectric yarns 576 in the 1×1 rib pattern structures B described above, and a plurality of openings 575 extending through the sample mesh interface 570 that are formed according the pointelle pattern structure C described above. The enlarged view of FIG. 17 is taken from the central portion 578 of the sample mesh interface 570. The sample mesh interface 570 also includes a peripheral portion 579 that includes the dielectric yarns 576 arranged in the tubular pattern structures A and the conductive yarn 577 loosely intertwined with the dielectric yarns 576 in the 1×1 rib pattern structures B.

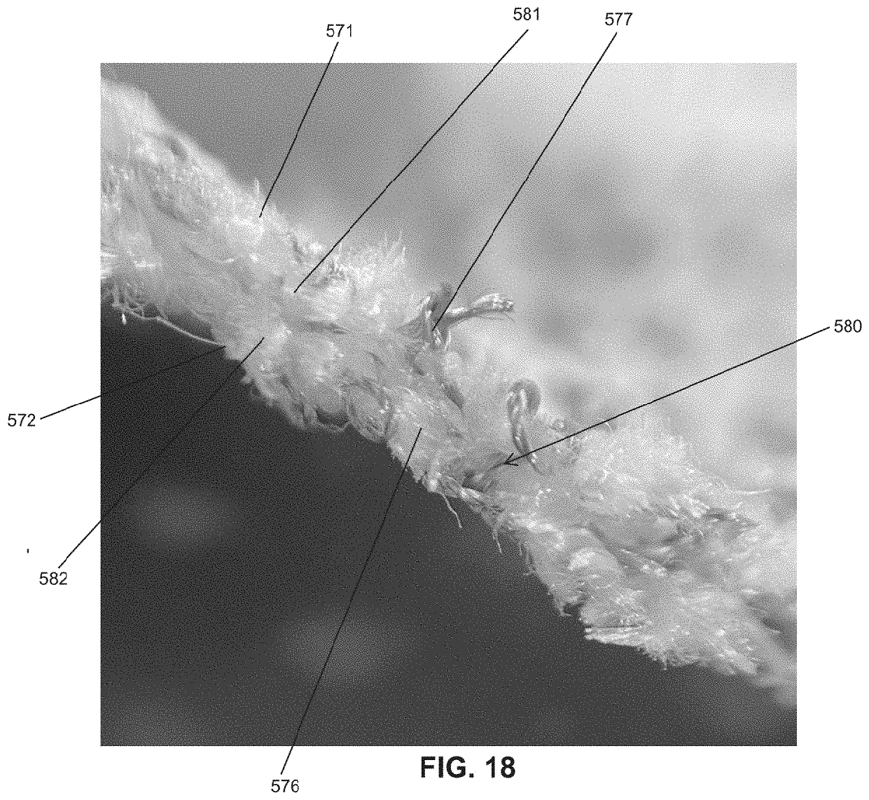

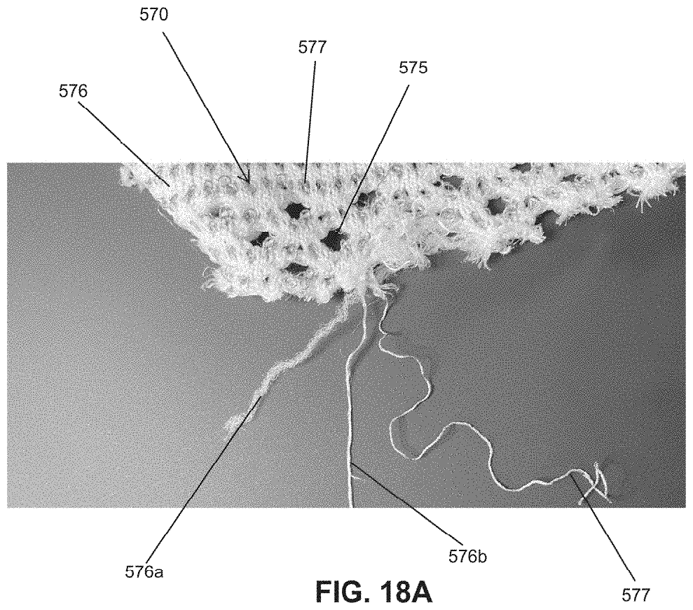

FIG. 18 illustrates an enlarged cross-sectional view of a portion of the sample mesh interface 570. The sample mesh interface 570 includes a first layer 581 and a second layer 582 of the dielectric yarns 576 knitted side by side (front and back), as discussed above. The conductive yarns 577 are intertwined through both layers 581, 582 of the dielectric yarns 576 to form the conductive pathways 580 extending through the sample mesh interface 570 from the first surface 571 of the sample mesh interface 570 to the second surface 572 of the sample mesh interface.

FIG. 18A illustrates an enlarged top view of a portion of the sample mesh interface 570. The knitting pattern structures in a portion of the sample mesh interface 570 have been unraveled to illustrate the individual yarns forming the sample mesh interface 570. As shown, the dielectric yarns 576 include a textured nylon yarn 576a and a fusible thermoplastic yarn 576b. The conductive yarn 577 is intertwined with the structures of the textured nylon yarn 576a and the fusible thermoplastic yarn 576b.

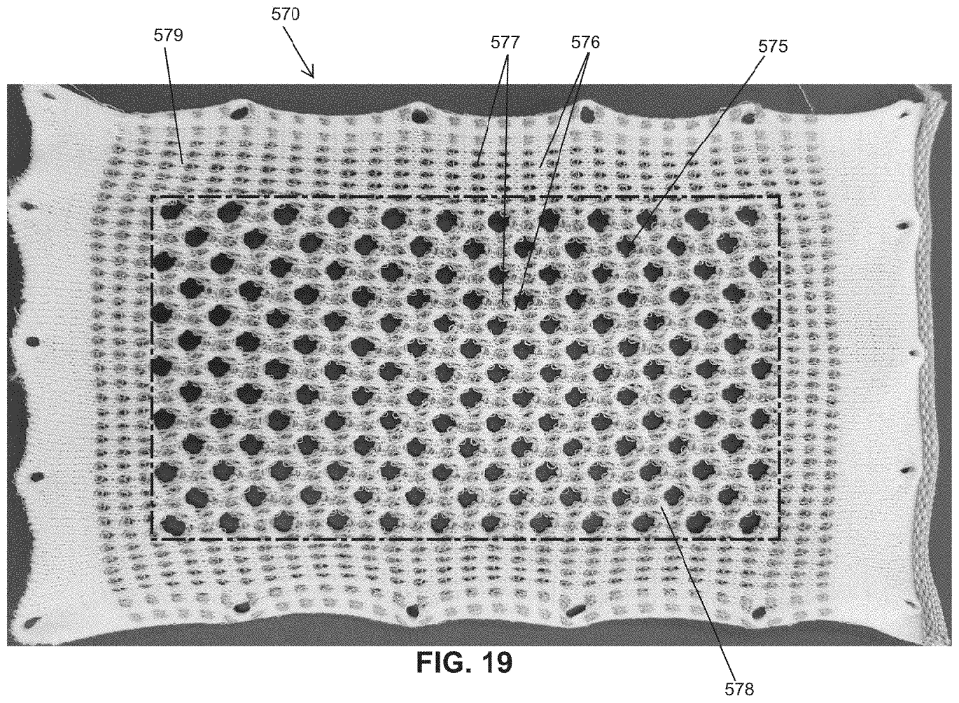

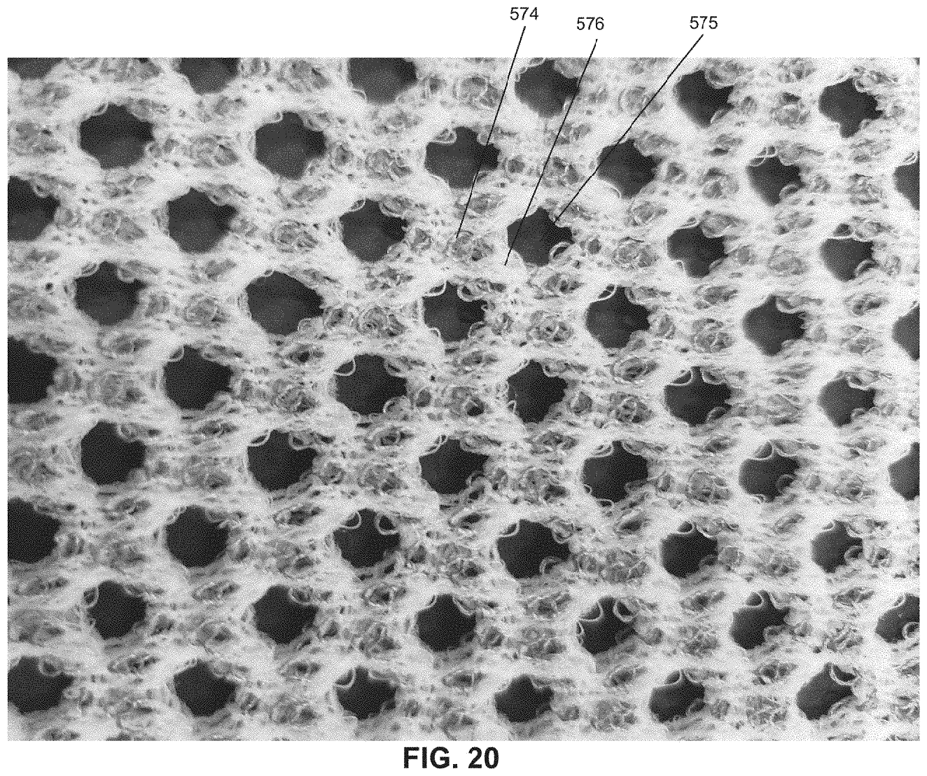

FIGS. 19-30 illustrate the sample mesh interface 570 after the sample mesh interface 570 has been stretched and exposed to heat in the form of a garment steamer set to apply heat at or above 75° C. FIG. 19 illustrates the sample mesh interface 570 after having been stretched and heated and shows the strands or fibers of the dielectric yarns having been contracted or fused/bonded together more tightly as a result of the exposure to heat. FIG. 20 illustrates an enlarged portion of the sample mesh interface 570 shown in FIG. 19. As shown, the sample mesh interface 570 has been exposed to heat such that the fusible thermoplastic yarn has melted, dissipated, and/or shrunk in volume, which has caused the above-described tubular pattern structures A of the dielectric yarns 576 to contract in volume relative to the conductive yarns 577 and the strands or fibers of the dielectric yarns 576 to be fused/bonded together more tightly. The contraction of the dielectric yarns 576 has resulted in the widening of the plurality of openings 575 in the central portion 578 of the sample mesh interface 570 and has also resulted in the conductive fibers 577 becoming fused more tightly within the dielectric yarns 576 and standing out more from the dielectric yarns 576.

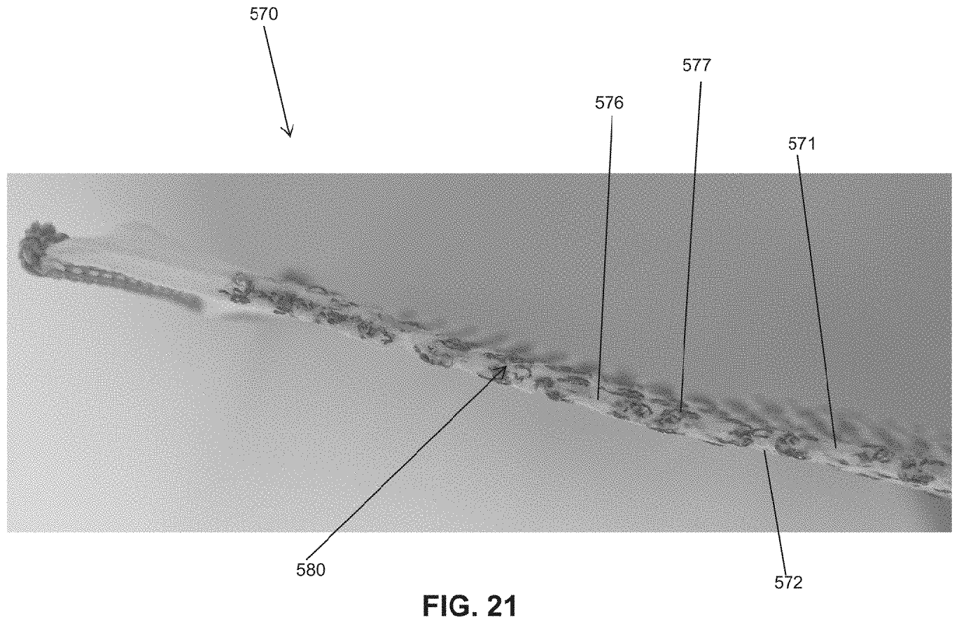

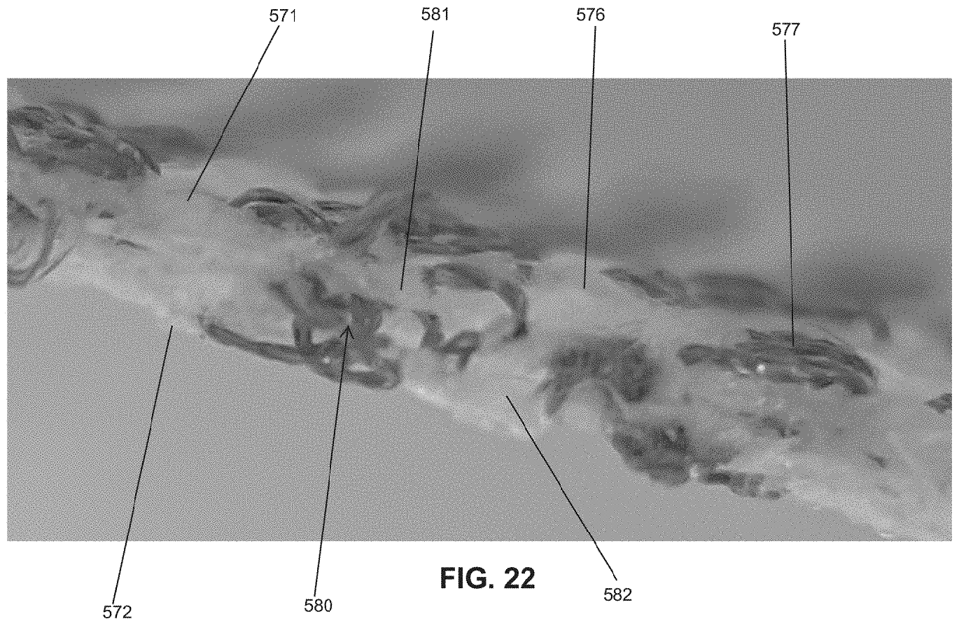

FIGS. 21 and 22 illustrate a side view and an enlarged portion of the side view of the sample mesh interface 570, respectively, after the stretching and exposure to heat. As shown, the fibers of the dielectric yarns 576 have become fused/bonded more tightly and contracted together with respect to the conductive yarns 577 and the layers 581, 582 of the dielectric yarns 576 have become more compacted. As a result, the conductive yarns 577 have been fused more tightly within the dielectric yarns 576 and stand out more from the dielectric yarns 576 such that the conductive pathways 580 extending through the sample mesh interface 570 from the first side 571 to the second side 572 project from the first surface 571 and the second surface 572 of the sample mesh interface 570.

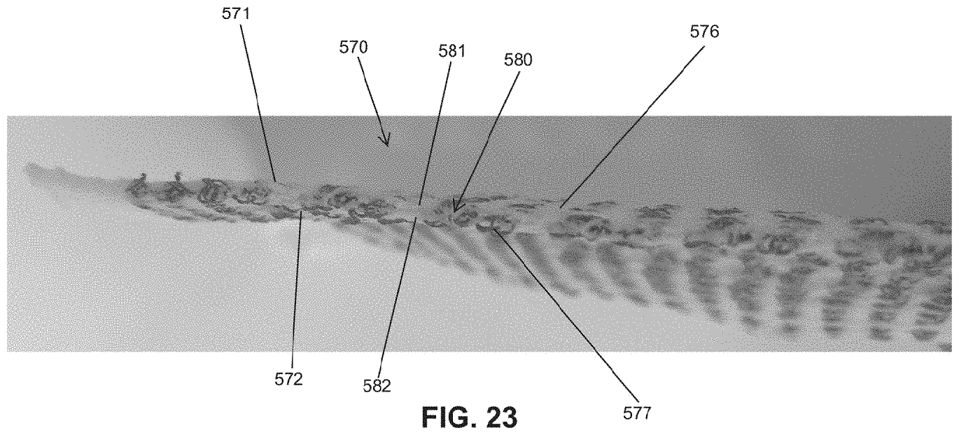

FIGS. 23-30 provide additional side views and enlarged partial side views of the sample mesh interface 570 after the stretching and exposure to heat. These views are provided as a supplement to the details shown in FIGS. 21 and 22.

Aspects of the present disclosure are directed to monitoring and/or therapeutic medical devices configured to identify a patient physiological event and, in response to the identified event, to provide a notification to the patient wearing the device. The notification can include an instruction or request to perform a patient response activity. Successful completion of the patient response activity can cause the device to suspend or delay a device function, such as administering a treatment to a patient and/or issuing an alert or alarm.

In some examples, the medical device includes monitoring circuitry configured to sense physiological information of a patient. The controller can be configured to detect the patient physiological event based, at least in part, on the sensed physiological information. A patient event can be a temporary physiological problem or abnormality, which can be representative of an underlying patient condition. A patient event can also include injuries and other non-recurring problems that are not representative of underlying physiological condition of the patient. A non-exhaustive list of patient events that can be detected by an external medical device includes, for example: bradycardia, ventricular tachycardia (VT) or ventricular fibrillation (VF), atrial arrhythmias such as premature atrial contractions (PACs), multifocal atrial tachycardia, atrial flutter, and atrial fibrillation, supraventricular tachycardia (SVT), junctional arrhythmias, tachycardia, junctional rhythm, junctional tachycardia, premature junctional contraction, and ventricular arrhythmias such as premature ventricular contractions (PVCs) and accelerated idioventricular rhythm.

In some examples, the device controller is configured to notify the patient of the detection of the one or more events and to receive a patient response to the notification. The patient response can include performing a response activity identifiable by an input component associated with the medical device. In general, the response activity is selected to demonstrate or to provide information about the status of the patient and, in particular, to confirm that the patient is conscious and substantially aware of his or her surroundings. The response activity or activities can also be configured to confirm patient identity (e.g., that the person providing the response is the patient, rather than a bystander or impostor). The response activity can also demonstrate or test a patient ability such as one or more of psychomotor ability, cognitive awareness, and athletic/movement ability. In some examples, the response activity can be a relatively simple action, such as making a simple or reflexive movement in response to a stimulus applied by the device. In other examples, more complex activities, such as providing answers to questions requiring reasoning and logical analysis can be required. The device can be configured to select a particular response activity based on characteristics of the patient and/or the detected patient event.

In some examples, the device can instruct the patient to perform several actions that are each representative of patient ability. In other modes, the device can instruct the patient to perform different types of activities that are representative of different patient abilities. For example, the device can instruct the patient to perform a single activity requiring several patient abilities to complete correctly. Alternatively, the device can instruct the patient to perform a first activity representative of a first patient ability and, once the first activity is correctly completed, to perform a second activity representative of a second patient ability.

This disclosure relates to components, modules, subsystems, circuitry, and/or techniques for use in external medical devices. For example, such components, modules, subsystems, circuitry, and/or techniques can be used in the context of medical devices for providing treatment to and/or monitoring a patient. For example, such medical devices can include monitoring devices configured to monitor a patient to identify occurrence of certain patient events. In some implementations, such devices are capable, in addition to monitoring for patient conditions, of providing treatment to a patient based on detecting a predetermined patient condition.

In some examples, the medical device can be a patient monitoring device, which can be configured to monitor one or more of a patient's physiological parameters without an accompanying treatment component. For example, a patient monitor may include a cardiac monitor for monitoring a patient's cardiac information. Such cardiac information can include, without limitation, heart rate, ECG data, heart sounds data from an acoustic sensor, and other cardiac data. In addition to cardiac monitoring, the patient monitor may perform monitoring of other relevant patient parameters, including glucose levels, blood oxygen levels, lung fluids, lung sounds, and blood pressure.

FIGS. 12-14 illustrate an example wearable medical device 100, such as a wearable defibrillator, which may incorporate the example support garment 50 discussed above with reference to FIGS. 4A-11B.

Non-limiting examples of suitable wearable defibrillators are disclosed in U.S. Pat. Nos. 4,928,690; 5,078,134; 5,741,306; 5,944,669; 6,065,154; 6,253,099; 6,280,461; 6,681,003; 8,271,082; and 8,369,944, the disclosure of each of which is hereby incorporated by reference. The wearable medical device 100 includes a plurality of sensing electrodes 112 that can be disposed at various positions about the patient's body. The sensing electrodes 112 are electrically coupled to a medical device controller 120 through a connection pod 130. In some implementations, some of the components of the wearable medical device 100 are affixed to a garment 110 that can be worn on the patient's torso. According to an example of the present disclosure, the garment 110 shown in FIG. 12 may be the same as the support garment 50 discussed above with reference to FIGS. 4A-11B.

The devices described herein are capable of continuous, substantially continuous, long-term and/or extended use or wear by, or attachment or connection to, a patient. In this regard, the device may be configured to be used or worn by, or attached or connected to, a patient, without substantial interruption, for example, up to hours or beyond (e.g., weeks, months, or even years). For example, in some implementations, such a period of use or wear may be at least 4 hours. For example, such a period of use or wear may be at least 24 hours or one day. For example, such a period of use or wear may be at least 7 days. For example, such a period of use or wear may be at least one month. In some implementations, such devices may be removed for a period of time before use, wear, attachment, or connection to the patient is resumed, e.g., to change batteries, to change or wash the garment, and/or to take a shower. Similarly, the device may be configured for continuous, substantially continuous, long-term and/or extended monitoring of one or more patient physiological conditions. For instance, in addition to cardiac monitoring, the medical device may be capable of monitoring a patient for other physiological conditions. Accordingly, in implementations, the device may be configured to monitor blood oxygen, temperature, glucose levels, sleep apnea, snoring and/or other sleep conditions, heart sounds, lung sounds, tissue fluids, etc. using a variety of sensors including radio frequency (RF) sensors, ultrasonic sensors, electrodes, etc. In some instances, the device may carry out its monitoring in periodic or aperiodic time intervals or times. For example, the monitoring during intervals or times can be triggered by a patient action or another event. For example, one or more durations between periodic or aperiodic intervals or times can be patient and/or other non-patient user configurable.

For example, as shown in FIG. 12, the controller 120 can be mounted on a belt worn by the patient. The sensing electrodes 112 and connection pod 130 can be assembled or integrated into the garment 110 as shown. The sensing electrodes 112 are configured to monitor the cardiac function of the patient (e.g., by monitoring one or more cardiac signals of the patient). While FIG. 12 shows four sensing electrodes 112, additional sensing electrodes may be provided, and the plurality of sensing electrodes 112 may be disposed at various locations about the patient's body.

The wearable medical device 100 can also optionally include a plurality of therapy electrodes 114 that are electrically coupled to the medical device controller 120 through the connection pod 130. The therapy electrodes 114 are configured to deliver one or more therapeutic electrical pulses, such as therapeutic transcutaneous defibrillating shocks, transcutaneous pacing pulses, and/or TENS pulses, to the body of the patient if it is determined that such treatment is warranted. The connection pod 130 may include electronic circuitry and one or more sensors (e.g., a motion sensor, an accelerometer, etc.) that are configured to monitor patient activity. In some implementations, the wearable medical device 100 may be a monitoring-only device that omits the therapy delivery capabilities and associated components (e.g., the therapy electrodes 114). In some implementations, various treatment components may be packaged into various modules that can be attached or removed from the wearable medical device 100 as needed. As shown in FIG. 12, the wearable medical device 100 may include a patient interface pod 140 that is electrically coupled to, integrated in, and/or integrated with the patient interface of the medical device controller 120. For example, the patient interface pod 140 may include patient interface elements such as a speaker, a microphone responsive to patient input, a display, an interactive touch screen responsive to patient input, and/or physical buttons for input.

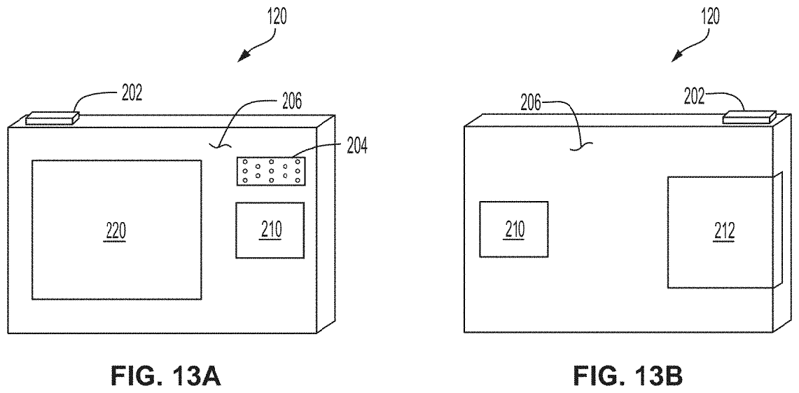

With reference to FIGS. 13A and 13B, an example of the medical device controller 120 is illustrated. The controller 120 may be powered by a rechargeable battery 212. The rechargeable battery 212 may be removable from a housing 206 of the medical device controller 120 to enable a patient and/or caregiver to swap a depleted (or near-depleted) battery 212 for a charged battery. The controller 120 includes a patient interface such as a touch screen 220 that can provide information to the patient, caregiver, and/or bystanders. In some implementations, in addition to or instead of a touch screen 220, the controller 120 may interact with the patient (e.g., receive patient input or provide information to the patient as described herein) via patient interface pod 140 (shown in FIG. 12). The patient interface pod 140 may be operatively coupled to the controller 120. In an example, the controller 120 may be configured to detect that if the patient interface pod 140 is operatively coupled to the controller 120, the controller 120 may then disable the patient interface elements of the controller 120 (e.g., touch screen 220) and instead communicate via the patient interface pod 140. The patient interface pod 140 may be wirelessly coupled with the controller 120. The patient interface pod 140 may take other forms and include additional functionality. For instance, the patient interface pod 140 may be implemented on a smartphone, tablet, or other mobile device carried by the patient. In another example, the patient interface pod 140 may be worn as a watch about the wrist of the patient, or as a band about an upper arm of the patient. In some implementations, the controller 120 may communicate certain alerts and information and/or be responsive to patient input via both the patient interface elements included in the controller 120 and the patient interface pod 140. The patient and/or caregiver can interact with the touch screen 220 or the patient interface pod 140 to control the medical device 100. The controller 120 also includes a speaker 204 for communicating information to the patient, caregiver, and/or the bystander. The controller 120 (and/or the patient interface pod 140) may include one or more response buttons 210. In some examples, when the controller 120 determines that the patient is experiencing cardiac arrhythmia, the speaker 204 can issue an audible alarm to alert the patient and bystanders to the patient's medical condition. In some examples, the controller 120 can instruct the patient to press one or both of the response buttons 210 to indicate that he or she is conscious, thereby instructing the medical device controller 120 to withhold the delivery of therapeutic defibrillating shocks. If the patient does not respond to an instruction from the controller 120, the medical device 100 may determine that the patient is unconscious and proceed with the treatment sequence, culminating in the delivery of one or more defibrillating shocks to the body of the patient. In some examples, as discussed in further detail herein, the controller 120 can additionally or alternatively instruct the patient to perform a response activity to indicate that he or she is conscious and further provide information to the controller 120 regarding the patient's status. For example, the controller 120 can instruct the patient to touch or manipulate the touch screen 220 or an interactive display on the patient interface pod 140 in a coordinated manner to confirm that he or she is conscious and has requisite awareness and/or psychomotor ability. In this way, the patient response confirms not only that buttons 210 were pressed, but that the patient is sufficiently conscious and aware to perform a response activity as instructed. The medical device controller 120 may further include a port 202 to removably connect sensing devices (e.g., ECG sensing electrodes 112) and/or therapeutic devices (e.g., therapy electrodes 114 shown in FIG. 12) to the medical device controller 120.

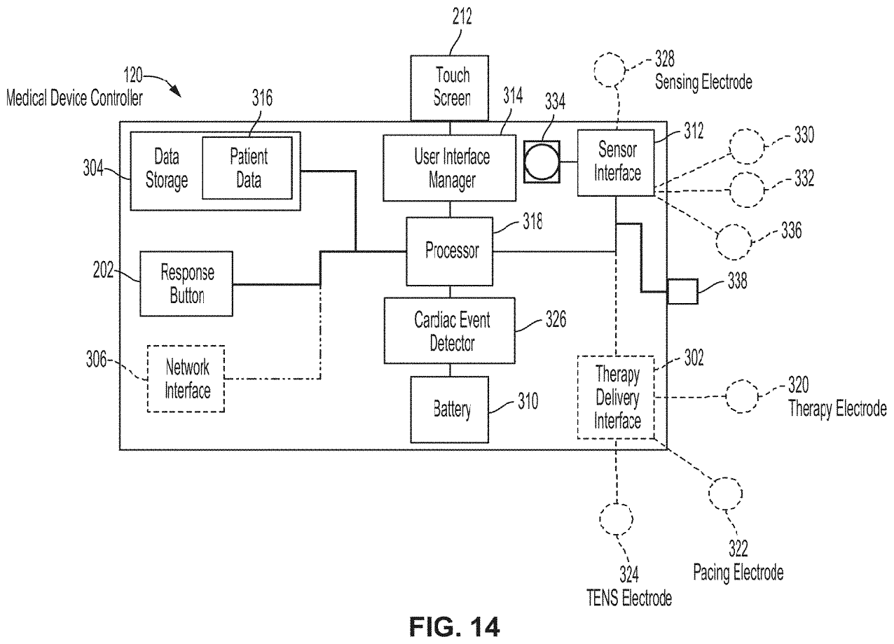

With reference to FIG. 14, a schematic example of the medical device controller 120 of FIGS. 12, 13A, and 13B is illustrated. As shown in FIG. 14, the controller 120 includes at least one processor 318, a patient interface manager 314, a sensor interface 312, an optional therapy delivery interface 302, data storage 304 (which may include patient data storage 316), an optional network interface 306, a patient interface 308 (e.g., including the touch screen 220 shown in FIGS. 13A and 13B), and a battery 310. The sensor interface 312 can be coupled to any one or combination of sensors to receive information indicative of cardiac activity. For example, the sensor interface 312 can be coupled to one or more sensing devices including, for example, sensing electrodes 328, contact sensors 330, pressure sensors 332, accelerometers or motion sensors 334, and radio frequency (RF)-energy based sensors 331 (e.g., tissue fluid sensors). The controller 120 can also include an optical sensor 336, such as a digital camera, for capturing static or video images of the device surroundings. Although designs from different vendors are different, a digital camera usually consists of a charge-coupled device (CCD) or complementary metal-oxide-semiconductor (CMOS) imaging sensor, a lens, a multifunctional video control chip, and a set of discrete components (e.g., capacitor, resistors, and connectors). The therapy delivery interface 302 (if included) can be coupled to one or more electrodes that provide therapy to the patient including, for example, one or more therapy electrodes 320, pacing electrodes 322, and/or TENS electrodes 324. The sensor interface 312 and the therapy delivery interface 302 may implement a variety of coupling and communication techniques for facilitating the exchange of data between the sensors and/or therapy delivery devices and the controller 120.

The medical device controller 120 may comprise one or more input components configured to receive a response input from the patient. The input components may comprise at least one of: the response button 210; the touch screen 220; an audio detection device, such as a microphone 338; the motion sensor 334; the contact sensor 330; the pressure sensor 332; a gesture recognitions component, such as the optical sensor 336; or a patient physiological sensor, such as the sensing electrodes 328.

In some examples, the medical device controller 120 includes a cardiac event detector 326 to monitor the cardiac activity of the patient and identify cardiac events experienced by the patient based on received cardiac signals. In other examples, cardiac event detection can be performed using algorithms for analyzing patient ECG signals obtained from the sensing electrodes 328. Additionally, the cardiac event detector 326 can access patient templates (e.g., which may be stored in the data storage 304 as patient data 316) that can assist the cardiac event detector 326 in identifying cardiac events experienced by the particular patient (e.g., by performing template matching algorithms).

The at least one processor 318 can perform a series of instructions that control the operation of the other components of the controller 120. In some examples, the patient interface manager 314 is implemented as a software component that is stored in the data storage 304 and executed by the at least one processor 318 to control, for example, the patient interface component 308. The patient interface manager 314 can control various output components and/or devices of the medical device controller 300 (e.g., patient interface 220 and/or patient interface pod 140 shown in FIG. 12) to communicate with external entities consistent with various acts and/or display screens described herein. For example, such output components and/or devices can include speakers, tactile and/or vibration output elements, visual indicators, monitors, displays, LCD screens, LEDs, Braille output elements, and the like. Additionally, the patient interface manager 314 can be integrated with the treatment-providing components of the controller 120 so that the patient can control and, in some cases, suspend, delay, or cancel treatment using the patient interface.

Although a wearable medical device and a support garment for such a device have been described in detail for the purpose of illustration based on what is currently considered to be the most practical examples, it is to be understood that such detail is solely for that purpose and that the subject matter of this disclosure is not limited to the disclosed examples, but, on the contrary, is intended to cover modifications and equivalent arrangements. For example, it is to be understood that this disclosure contemplates that, to the extent possible, one or more features of any example can be combined with one or more features of any other example.

1. A support garment for use with a wearable cardiac therapeutic device, the garment comprising:
a mesh interface configured to transmit therapeutic electrical pulses between at least one therapy electrode and a patient's skin, the mesh interface comprising:
a plurality of dielectric fibers comprising at least one nonmetallic material; and
a plurality of conductive fibers or particles interspersed with the plurality of dielectric fibers, the plurality of conductive fibers being configured to form a plurality of conductive pathways extending through the mesh interface,
wherein the plurality of conductive pathways are configured to conduct the therapeutic electrical pulses through the mesh interface from the at least one therapy electrode to the patient's skin.

2. The support garment according to claim 1, further comprising at least one support pocket disposed on an inside surface of the support garment, the support pocket being configured to support the at least one therapy electrode on the support garment,
wherein the mesh interface forms a part of the at least one support pocket.

3.-5. (canceled)

6. The support garment according to claim 1, wherein the mesh interface is configured to transmit a falloff signal from the at least one therapy electrode to the patient's skin configured to determine that the at least one therapy electrode is correctly positioned on the patient's body.

7. The support garment according to claim 1, wherein the mesh interface is configured to provide an electrical impedance of the plurality of conductive pathways extending through the mesh interface from the first surface to the second surface of approximately 0.01Ω-5Ω.

8. The support garment according to claim 1, wherein the plurality of conductive fibers or particles comprises an impedance measure of approximately 10-250 Ω/meter.

9. The support garment according to claim 1, wherein the mesh interface further comprises a plurality of openings extending through the mesh interface from the first surface to the second surface, the mesh interface being configured to facilitate transfer of conductive gel from the at least one therapy electrode to the patient's skin via the plurality of openings.

10. (canceled)

11. The support garment according to claim 9, wherein the conductive gel is configured to provide a predetermined electrical impendence of the plurality of conductive pathways extending through the mesh interface from the first surface to the second surface of approximately 0.01Ω-5Ω.

12. (canceled)

13. The support garment according to claim 1, wherein the mesh interface comprises a plurality of openings, the plurality of openings comprising approximately 2-1000 openings per square inch of the mesh interface.

14. The support garment according to claim 1, wherein the mesh interface comprises a plurality of openings having an average diameter in a range of approximately 0.005″-0.3″ (0.13 mm-7.6 mm).

15. The support garment according to claim 1, wherein the plurality of dielectric fibers of the mesh interface comprise a dielectric yarn and the plurality of conductive fibers or particles of the mesh interface comprise a conductive yarn, and wherein the dielectric yarn and the conductive yarn are intertwined together to form the mesh interface.

16.-47. (canceled)

48. A wearable cardiac therapeutic device for improved skin comfort when worn by a patient, the device comprising:
at least one therapy electrode configured to deliver therapeutic electrical pulses to a patient's heart; and
a support garment configured to support the at least one therapy electrode in electrical communication with the patient's body, the support garment comprising:
at least one support pocket disposed on an inside surface of the support garment for supporting the at least one therapy electrode on the support garment; and
a mesh interface formed as part of the at least one support pocket, the mesh interface configured to facilitate electrical contact between the at least one therapy electrode and the patient's skin,
wherein the mesh interface comprises:
a first surface oriented toward the at least one therapy electrode;
a second surface oriented toward the patient's skin;
a plurality of dielectric fibers comprising at least one nonmetallic material;
a plurality of conductive fibers or particles; and
a plurality of openings extending through the mesh interface from the first surface to the second surface, and
wherein the mesh interface is configured to facilitate a transfer of conductive gel from the at least one therapy electrode to the patient's skin via the plurality of openings.

49. (canceled)

50. The wearable cardiac therapeutic device according to claim 48, wherein the mesh interface is configured to transmit a falloff signal from the at least one therapy electrode to the patient's skin configured to determine that the at least one therapy electrode is correctly positioned on the patient's body.

51. The wearable cardiac therapeutic device according to claim 48, wherein the plurality of dielectric fibers and the plurality of conductive fibers or particles are interspersed to form a plurality of conductive pathways extending through the mesh interface from the first surface to the second surface, the plurality of conductive pathways being configured to conduct the therapeutic electrical pulses through the mesh interface from the at least one therapy electrode to the patient.

52. The wearable cardiac therapeutic device according to claim 51, wherein the mesh interface is configured to provide an electrical impedance of the plurality of conductive pathways extending through the mesh interface from the first surface to the second surface of approximately 0.01Ω-5Ω.

53. The wearable cardiac therapeutic device according to claim 51, wherein the plurality of conductive fibers or particles comprises an impedance measure of approximately 10-250 Ω/meter.

54.-55. (canceled)

56. The wearable cardiac therapeutic device according to claim 51, wherein the mesh interface is configured to be porous to the conductive gel from a plurality of holes in the at least one therapy electrode to provide a predetermined electrical impendence of the plurality of conductive pathways extending through the mesh interface from the first surface to the second surface of approximately 0.01Ω-5Ω.

57.-58. (canceled)

59. The wearable cardiac therapeutic device according to claim 48, wherein a thickness of the mesh interface from the first surface to the second surface is approximately 0.005″-0.5″ (0.13 mm-12.7 mm).

60. The wearable cardiac therapeutic device according to claim 48, wherein the dielectric fibers comprise, at least in part, fusible fibers that are configured to shrink in volume relative to the conductive fibers or particles when the mesh interface is exposed to heat.

61. (canceled)

62. The wearable cardiac therapeutic device according to claim 48, wherein the plurality of dielectric fibers of the mesh interface comprise a dielectric yarn and the plurality of conductive fibers or particles of the mesh interface comprise a conductive yarn, and wherein the dielectric yarn and the conductive yarn are intertwined together to form the mesh interface.

63.-65. (canceled)

66. The wearable cardiac therapeutic device according to claim 62, wherein the mesh interface comprises a plurality of intertwined structures of the dielectric yarn and the conductive yarn.

67.-75. (canceled)
